(19) United States
(12) Patent Application Publication
(10) Pub. No.: US 2022/0152396 A1
(43) Pub. Date: May 19, 2022

(54) METHOD, DEVICE AND SYSTEM FOR SIMULATING AND/OR DELIVERING DEEP BRAIN STIMULATION

(71) Applicant: University Health Network, Toronto (CA)

(72) Inventors: Luka Milosevic, Hamilton (CA); Milos R. Popovic, Mississauga (CA); Milad Lankarany, Toronto (CA); William D. Hutchison, Toronto (CA)

(21) Appl. No.: 17/527,042

(22) Filed: Nov. 15, 2021

Related U.S. Application Data
(60) Provisional application No. 63/198,795, filed on Nov. 13, 2020.

(51) Int. Cl. A61N 1/36; A61N 1/05

(52) U.S. Cl. CPC A61N 1/36082; A61N 1/36128; A61N 1/0534

(57) ABSTRACT

Various embodiments are described herein for a device, system and method for delivering deep brain stimulation of a target structure. A target model corresponding to the target structure is used where the model includes a neuronal model for modeling one or more neurons of the target structure and a short-term plasticity model for modeling temporal behaviour of the neuron in response to one or more stimulus pulses over time. The method generally includes receiving input data related to the target structure including a brain region of the target structure, stimulus parameters including pulse frequency, and model parameters that correspond to the brain structure; determining a neuronal response of the target structure by applying the stimulus parameters and the model parameters to the target model; and performing any combination of outputting the neuronal response, storing the neuronal response and transmitting the neuronal response to another device.

### CROSS-REFERENCE

This application claims the benefit of U.S. Provisional Patent Application No. 63/198,795, filed Nov. 13, 2020, and the entire contents of U.S. Provisional Patent Application No. 63/198,795 is hereby incorporated by reference.

### FIELD

The present disclosure relates generally to neurostimulation; and more particularly to a system and a method for simulating and/or delivering a deep brain stimulation.

### BACKGROUND

Deep brain stimulation (DBS) is a neuromodulatory therapy that may be used to treat several movement disorders such as, but not limited to, Parkinson's disease, essential tremor, dystonia; or other neurological conditions such as, but not limited to, Alzheimer's, obsessive-compulsive disorder and epilepsy, for example. However, despite a rapidly growing interest in the development of DBS indications, there is a lack of understanding of the physiological effects elicited by DBS has hindered its expansion beyond early success in movement disorders.

### SUMMARY OF VARIOUS EMBODIMENTS

In one broad aspect, in accordance with the teachings herein, there is provided a device for performing one or more functions related to deep brain stimulation (DBS) of a target structure, wherein the device comprises: a memory unit storing program instructions for performing one or more functions related to DBS and a target model corresponding to the target structure, the target model including a neuronal model for modeling one or more neurons of the target structure and a short-term plasticity model for modeling temporal behaviour of the neuron in response to one or more stimulus pulses over time; one or more processors that are coupled to the memory unit, the one or more processors, when executing the program instructions, being configured to: receive input data related to the target structure including a brain region of the target structure, stimulus parameters including pulse frequency, and model parameters that correspond to the brain structure; determine a neuronal response of the target structure by applying the stimulus parameters and the model parameters to the target model; and perform any combination of outputting the neuronal response, storing the neuronal response and transmitting the neuronal response to another device.

In at least one embodiment, the model parameters include a proportion of inhibitory inputs and exhibitory inputs for the target structure.

In at least one embodiment, the neuron model comprises a leaky integrate and fire (LIF) single neuron model.

In at least one embodiment, the short-term plasticity model comprises the Tsodyks-Markram (TM) model.

In at least one embodiment, the target model further a background synaptic activity model that is added to the neuron model to reproduce an impact of synaptic noise, the background synaptic activity model being implemented using an Ornstein-Uhlenbeck process.

In at least one embodiment, the memory unit stores determination program instructions that, when executed by the one or more processors, configure the one or more processors for determining one or more of the stimulus parameters, including frequency, generating inhibitory and excitatory synaptic responses and scaling the inhibitory and excitatory synaptic responses with respect to weights representing the proportion of inhibitory and exhibitory inputs.

In at least one embodiment, the memory unit stores stimulation program instructions that, when executed by the one or more processors, configure the one or more processors for controlling one or more stimulation device(s) to deliver DBS to a patient where stimulus frequency is selected based on the target structure and desired treatment for the patient.

In at least one embodiment, when there are more inhibitory inputs than exhibitory inputs associated with the target structure, the one or more processors, when executing the stimulation instructions, are configured to: control the one or more stimulation devices to deliver a low frequency stimulation when partial suppression of neuronal output is desired; or control the one or more stimulation devices to deliver a high frequency stimulation when complete suppression of neuronal output is desired.

In at least one embodiment, the when there are more exhibitory inputs than inhibitory inputs associated with the target structure, the one or more processors, when executing the stimulation instructions, are configured to: control the one or more stimulation devices to deliver a low frequency stimulation when upregulation of neuronal activity is determined; or control the one or more stimulation devices to deliver a high frequency stimulation when downregulation of neuronal activity is desired.

In at least one embodiment, the DBS stimulus is provided to the patient using an open loop stimulation mode or a closed loop stimulation mode.

In another broad aspect, in accordance with the teachings herein, there is provided at least one embodiment of a method for performing one or more functions related to deep brain stimulation (DBS) of a target structure, wherein the method is performed by one or more processors and the method comprises obtaining a target model corresponding to the target structure, the target model including a neuronal model for modeling one or more neurons of the target structure and a short-term plasticity model for modeling temporal behaviour of the neuron in response to one or more stimulus pulses over time; receiving input data related to the target structure including a brain region of the target structure, stimulus parameters including pulse frequency, and model parameters that correspond to the brain structure; determining a neuronal response of the target structure by applying the stimulus parameters and the model parameters to the target model; and performing any combination of outputting the neuronal response, storing the neuronal response and transmitting the neuronal response to another device.

In at least one embodiment, the method comprises determining one or more of the stimulus parameters, including frequency, generating inhibitory and excitatory synaptic responses and scaling the inhibitory and excitatory synaptic responses with respect to weights representing the proportion of inhibitory and exhibitory inputs.

In at least one embodiment, the method comprises controlling one or more stimulation device(s) to deliver DBS to a patient where stimulus frequency is selected based on the target structure and desired treatment for the patient and when there are more inhibitory inputs than exhibitory inputs associated with the target structure, the method further comprises: controlling the one or more stimulation devices to deliver a low frequency stimulation when partial suppression of neuronal output is desired; or controlling the one or more stimulation devices to deliver a high frequency stimulation when complete suppression of neuronal output is desired.

In at least one embodiment, the method comprises controlling one or more stimulation device(s) to deliver DBS to a patient where stimulus frequency is selected based on the target structure and desired treatment for the patient and when there are more exhibitory inputs than inhibitory inputs associated the method further comprises: controlling the one or more stimulation devices to deliver a low frequency stimulation when upregulation of neuronal activity is desired; or controlling the one or more stimulation devices to deliver a high frequency stimulation when downregulation of neuronal activity is desired.

In another broad aspect, in accordance with the teachings herein, there is provided at least one embodiment of a computer readable medium comprising a plurality of instructions that are executable on one or more processors of a device for configuring the one or more processors to implement a method for at least one function related to deep brain stimulation, wherein the method is defined according to any of the embodiments described herein.

In another broad aspect, in accordance with the teachings herein, there is provided at least one embodiment of a method for delivering deep brain stimulation, where the method comprises: receiving input data for a target structure; determining whether there are more inhibitory or exhibitory inputs associated with the received target structure; where there are more inhibitory inputs: determining whether to partially or completely suppress neuronal output; where partial suppression of neuronal output is determined, delivering a low frequency stimulation; and where complete suppression of neuronal output is determined, delivering a high frequency stimulation; and where there are more exhibitory inputs: determining whether to upregulate or downregulate neuronal activity; where upregulation of neuronal activity is determined, delivering a low frequency stimulation; and where downregulation of neuronal activity is determined, delivering a high frequency stimulation.

According to another broad aspect of the teachings herein, in at least one embodiment described herein there is provided a deep brain stimulator and a system and method for delivering deep brain stimulation. The method comprising: receiving input data for a target structure; determining whether there are more inhibitory or exhibitory inputs associated with the received target structure; where there are more inhibitory inputs: determining whether to partially or completely suppress neuronal output; where partial suppression of neuronal output is determined, delivering a low frequency stimulation; and where complete suppression of neuronal output is determined, delivering a high frequency stimulation; and where there are more exhibitory inputs: determining whether to upregulate or downregulate neuronal activity; where upregulation of neuronal activity is determined, delivering a low frequency stimulation; and where downregulation of neuronal activity is determined, delivering a high frequency stimulation.

It will be appreciated that the foregoing summary sets out representative aspects of embodiments to assist skilled readers in understanding the following detailed description. Other features and advantages of the present application will become apparent from the following detailed description taken together with the accompanying drawings. It should be understood, however, that the detailed description and the specific examples, while indicating preferred embodiments of the application, are given by way of illustration only, since various changes and modifications within the spirit and scope of the application will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 shows a block diagram of an example embodiment of a system for predicting neural responses (e.g., excitation/inhibition/suppression) and/or providing stimuli for deep brain stimulation, in accordance with the teachings herein.

FIG. 2A shows a flowchart of an example embodiment of a method for predicting neural responses (e.g., excitation/inhibition/suppression) and/or providing stimuli for deep brain stimulation, in accordance with the teachings herein.

FIG. 2B shows a flowchart of an example embodiment of a method for delivering deep brain stimulation, in accordance with the teachings herein.

FIG. 3 shows peristimulus responses to single stimulation pulses obtained in an experimental study.

FIG. 4 shows stimulation frequency response functions and sagittal sections depicting the locations of investigated structures in the experimental study.

FIG. 5A shows time domain responses to stimulation trains that were obtained during the experimental study.

FIG. 5B shows time-domain firing histograms for short trains of 50 Hz and 100 Hz where in Vim and Rt, a decay of neuronal excitation (mean+standard error) also occurred with short trains of 50 Hz and 100 Hz stimulation (50 pulses each).

FIG. 5C shows examples of induction of neural oscillations with low-frequency stimulation in the Vim and SNr structures.

FIG. 6 shows diagrams of models for modelling single pulse responses that may be used by the system and method of FIGS. 1 to 2B.

FIG. 7 shows simulated responses to single stimulation pulses and peristimulus firing rate histograms that were determined using an example embodiment of a model framework in accordance with the teachings herein.

FIG. 8 shows simulated synaptic currents with short-term plasticity that were determined using an example embodiment of a model framework in accordance with the teachings herein.

FIG. 9 shows simulated and real voltages for Vim in response to stimulation at different frequencies where the simulated voltages are determined using an example embodiment of a model framework in accordance with the teachings herein.

FIG. 10 shows simulated and real voltages for STN in response to stimulation at different frequencies where the simulated voltages are determined using an example embodiment of a model framework in accordance with the teachings herein.

FIG. 11 shows simulated and real voltages for SNr in response to stimulation at different frequencies where the simulated voltages are determined using an example embodiment of a model framework in accordance with the teachings herein.

FIG. 12 shows computational frequency response functions for Vim, STN and SNr for firing rate versus stimulus frequency determined using an example embodiment of a model framework in accordance with the teachings herein.

Further aspects and features of the example embodiments described herein will appear from the following description taken together with the accompanying drawings.

### DETAILED DESCRIPTION OF THE EMBODIMENTS

The headings and Abstract of the Disclosure provided herein are for convenience only and do not interpret the scope or meaning of the embodiments.

Various embodiments in accordance with the teachings herein will be described below to provide examples of at least one embodiment of the claimed subject matter. No embodiment described herein limits any claimed subject matter. The claimed subject matter is not limited to devices, systems or methods having all of the features of any one of the devices, systems or methods described below or to features common to multiple or all of the devices, systems or methods described herein. It is possible that there may be a device, system or method described herein that is not an embodiment of any claimed subject matter. Any subject matter that is described herein that is not claimed in this document may be the subject matter of another protective instrument, for example, a continuing patent application, and the applicants, inventors or owners do not intend to abandon, disclaim or dedicate to the public any such subject matter by its disclosure in this document.

It will be appreciated that for simplicity and clarity of illustration, where considered appropriate, reference numerals may be repeated among the Figures to indicate corresponding or analogous elements or steps. In addition, numerous specific details are set forth in order to provide a thorough understanding of the embodiments described herein. However, it will be understood by those of ordinary skill in the art that the embodiments described herein may be practised without these specific details. In other instances, well-known methods, procedures and components have not been described in detail so as not to obscure the embodiments described herein. Also, the description is not to be considered as limiting the scope of the embodiments described herein.

It should also be noted that the terms “coupled”, or “coupling” as used herein can have several different meanings depending in the context in which these terms are used. For example, the terms coupled, or coupling can have a mechanical or electrical connotation. For example, as used herein, the terms coupled or coupling can indicate that two elements or devices can be directly connected to one another or connected to one another through one or more intermediate elements or devices via an electrical or magnetic signal, electrical connection, an electrical element or a mechanical element depending on the particular context. Furthermore, certain coupled electrical elements may send and/or receive data.

Unless the context requires otherwise, throughout the specification and claims which follow, the word “comprise” and variations thereof, such as, “comprises” and “comprising” are to be construed in an open, inclusive sense, that is, as “including, but not limited to”.

Various terms used throughout the present description may be read and understood as follows, unless the context indicates otherwise: singular articles and pronouns as used throughout include their plural forms, and vice versa; similarly, gendered pronouns include their counterpart pronouns so that pronouns should not be understood as limiting anything described herein to use, implementation, performance, etc. by a single gender. Further definitions for terms may be set out herein; these may apply to prior and subsequent instances of those terms, as will be understood from a reading of the present description.

It should also be noted that, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

It should be noted that terms of degree such as “substantially”, “about” and “approximately” as used herein mean a reasonable amount of deviation of the modified term such that the end result is not significantly changed. These terms of degree may also be construed as including a deviation of the modified term, such as by 1%, 2%, 5% or 10%, for example, if this deviation does not negate the meaning of the term it modifies.

Furthermore, the recitation of numerical ranges by endpoints herein includes all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed, such as 1%, 2%, 5%, or 10%, for example.

Reference throughout this specification to “one embodiment”, “an embodiment”, “at least one embodiment” or “some embodiments” means that one or more particular features, structures, or characteristics may be combined in any suitable manner in one or more embodiments, unless otherwise specified to be not combinable or to be alternative options.

As used in this specification and the appended claims, the singular forms “a,” “an,” and “the” include plural referents unless the content clearly dictates otherwise. It should also be noted that the term “or” is generally employed in its broadest sense, that is, as meaning “and/or” unless the content clearly dictates otherwise.

Similarly, throughout this specification and the appended claims the term “communicative” as in “communicative pathway,” “communicative coupling,” and in variants such as “communicatively coupled,” is generally used to refer to any engineered arrangement for transferring and/or exchanging information. Examples of communicative pathways include, but are not limited to, electrically conductive pathways (e.g., electrically conductive wires, physiological signal conduction), electromagnetically radiative pathways (e.g., radio waves), or any combination thereof. Examples of communicative couplings include, but are not limited to, electrical couplings, magnetic couplings, radio couplings, or any combination thereof.

Throughout this specification and the appended claims, infinitive verb forms are often used. Examples include, without limitation: “to detect,” “to provide,” “to transmit,” “to communicate,” “to process,” “to route,” and the like. Unless the specific context requires otherwise, such infinitive verb forms are used in an open, inclusive sense, that is as “to, at least, detect,” to, at least, provide,” “to, at least, transmit,” and so on.

A portion of the example embodiments of the systems, devices, or methods described in accordance with the teachings herein may be implemented as a combination of hardware or software. For example, a portion of the embodiments described herein may be implemented, at least in part, by using one or more computer programs, executing on one or more programmable devices comprising at least one processing element, and at least one data storage element (including volatile and non-volatile memory). These devices may also have at least one input device (e.g., a keyboard, a mouse, a touchscreen, and the like) and at least one output device (e.g., a display screen, a printer, a wireless radio, and the like) depending on the nature of the device.

It should also be noted that there may be some elements that are used to implement at least part of the embodiments described herein that may be implemented via software that is written in a high-level procedural language such as object-oriented programming. The program code may be written in C, C⁺⁺ or any other suitable programming language and may comprise modules or classes, as is known to those skilled in object-oriented programming. Alternatively, or in addition thereto, some of these elements implemented via software may be written in assembly language, machine language, or firmware as needed.

At least some of the software programs used to implement at least one of the embodiments described herein may be stored on a storage media or a device that is readable by a general or special purpose programmable device. The software program code, when read by the programmable device, configures the programmable device to operate in a new, specific and predefined manner in order to perform at least one of the methods described herein.

Furthermore, at least some of the programs associated with the systems and methods of the embodiments described herein may be capable of being distributed in a computer program product comprising a computer readable medium that bears computer usable instructions, such as program code, for one or more processors. The program code may be preinstalled and embedded during manufacture and/or may be later installed as an update for an already deployed computing system. The medium may be provided in various forms, including non-transitory forms such as, but not limited to, one or more diskettes, compact disks, tapes, chips, and magnetic and electronic storage. In alternative embodiments, the medium may be transitory in nature such as, but not limited to, wire-line transmissions, satellite transmissions, internet transmissions (e.g., downloads), media, digital and analog signals, and the like. The computer useable instructions may also be in various formats, including compiled and non-compiled code.

Accordingly, any module, unit, component, server, computer, terminal or device described herein that executes software instructions may include or otherwise have access to computer readable media such as storage media, computer storage media, or data storage devices (removable and/or non-removable) such as, for example, magnetic disks, optical disks, or tape. Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information, and which can be accessed by an application, module, or both. Any such computer storage media may be part of the device or accessible or connectable thereto.

Further, unless the context clearly indicates otherwise, any processor or controller set out herein may be implemented as a singular processor or as a plurality of processors. The plurality of processors may be arrayed or distributed, and any processing function referred to herein may be carried out by one or by a plurality of processors, even though a single processor may be exemplified. Any method, software application or software module herein described may be implemented using computer readable/executable instructions that may be stored or otherwise held by such computer readable media and executed by the one or more processors.

Generally, physiological understandings of the effects of deep brain stimulation are very limited. Current approaches generally do not develop paradigms with consideration to how the stimulation is influencing neuronal activity but rather are based on an empirical “broad parameter screening” approach where a clinician arbitrarily chooses a frequency and applies it to gauge its effects. For example, in treatments for Alzheimer's or epilepsy, there is no way to know if such parameters were clinically effective without applying stimuli having certain stimulus parameters, sending the patient home and reassessing them several months later to see if the stimulus parameters used during treatment were beneficial. If it was not effective, some stimulus parameters are changed, the treatment is re-applied, the patient is sent home, and then reassessed again a few months later.

In contrast, in accordance with the teachings herein, in one aspect, there is provided at least one embodiment of a system and a method that may be used to determine how stimulation, using certain frequency parameters, influences neuronal activity in a target structure such as a certain region of the brain, with which hypothesis-driven approaches to DBS can be employed, without the completely arbitrary nature of the above empirical approach. A target model, as described herein, can instead be used to allow users to determine the desired (hypothesis-driven) changes to the brain activity.

This is useful since pathophysiologically-informed target selection and subsequent stimulation programming may play important roles in the development of novel indications for deep brain stimulation (DBS). Furthermore, generally the effects of electrical stimulation in one area of the brain may differ from the effects of electrical stimulation in another area of the brain. Accordingly, in at least one embodiment described herein, the varying effects of DBS at the neuronal level depending on where and how electrical stimulation is applied is taken into account.

For example, single pulses of electrical stimulation delivered to the substantia nigra pars reticulata (SNr), which is an emerging DBS target for intractable axial motor symptoms of Parkinson's disease, or to the globus pallidus internus (Gpi), which is a conventional target for Parkinson's disease and dystonia, have been associated with stimulation-evoked inhibitory responses, likely mediated by local GABA (γ-amino-butyric acid) release. In contrast, high-frequency stimulation (HFS) of the thalamic ventral intermediate nucleus (Vim) elicited brief short-latency excitatory responses, likely the result of local glutamate release.

In another aspect, the inventors have determined that parameters for effective stimuli for treating certain neural conditions can incorporate the effects of individual electrical stimulation pulses which vary with respect to the distribution of afferent inputs converging on target neurons (whether predominantly inhibitory or excitatory), and that based on these local neuroanatomical properties, stimulation pulses elicit either net inhibitory responses or net excitatory responses. Furthermore, the inventors have determined that stimulus-evoked excitatory responses are generally attenuated with increasing stimulation frequencies, i.e., high-Frequency Stimulation (HFS). HFS can reduce synaptic transmission fidelity through synaptic depression or axonal failure. The inventors have determined that this may be modelled using short-term synaptic plasticity.

Therefore, in at least one embodiment described in accordance with the teachings herein, a modelling framework is used, that has been developed by the inventors, and includes combining different models to account for different neural effects as well as use different parameters for different areas of the brain for the prediction of site-specific and frequency-dependent neuronal responses to electrical stimulation. Individual DBS pulses (i.e., extracellular stimulation) initiate action potentials which are propagated with high fidelity along the axons of stimulated neurons including both efferent axons and afferent axons and/or their terminals. The modelling framework may also be implemented to account for anatomical differences across stimulation sites which may incur dissimilar post-synaptic responses (i.e., neuronal excitation vs. inhibition). In addition, the modelling framework may also incorporate one or more models, such as the Tsodyks-Markram (TM) model, for example, to model short-term synaptic plasticity (i.e., frequency-dependent physiological properties) in order to account for changes to synaptic transmission fidelity based on the frequency of successive stimuli.

In another aspect, in at least one embodiment described herein, the modelling framework may be implemented to determine post-synaptic neuronal responses to DBS pulses that are a result of the simultaneous activation of presynaptic inputs, and furthermore, take into consideration proportions of inhibitory and excitatory inputs converging on target neurons (derived from anatomical/morphological data). Advantageously, at least one embodiment described herein takes into account biophysical mechanisms underlying DBS at different frequencies across various brain regions within a particular framework, which may be used in the context of the human brain.

In another aspect, in accordance with the teachings herein, at least one embodiment is provided that may be used to predict neuronal response to electrical stimulation pulses on a region-to-region basis; determining whether stimulation will upregulate or downregulate neuronal output such as, for example, to determine whether to upregulate brain activity or downregulate brain activity in a defined functional manner by, e.g., increasing or decreasing communication efficacy across brain regions. This prediction can be determinative of brain-region-specific and/or frequency-specific stimulation effects.

In another aspect, in at least one embodiment provided in accordance with the teachings herein, synaptic transmission fidelity (i.e., synaptic filter properties) may be predicted with changes to stimulation parameters; for example, to switch from upregulation to downregulation of neuronal activity with changes to stimulation frequency. For example, the target model framework described herein allows for synaptic dynamics to be changed in a computer model in order to predict how different stimulation parameters change the firing pattern of a neuron.

In another aspect, at least one embodiment described in accordance with the teachings herein may be used to determine anatomical properties (for example, proportions of inhibitory and excitatory inputs) based on the response to single stimulation pulses. This can have diagnostic and/or prognostic implications and can be used to determine a desired functional neuronal outcome of stimulation.

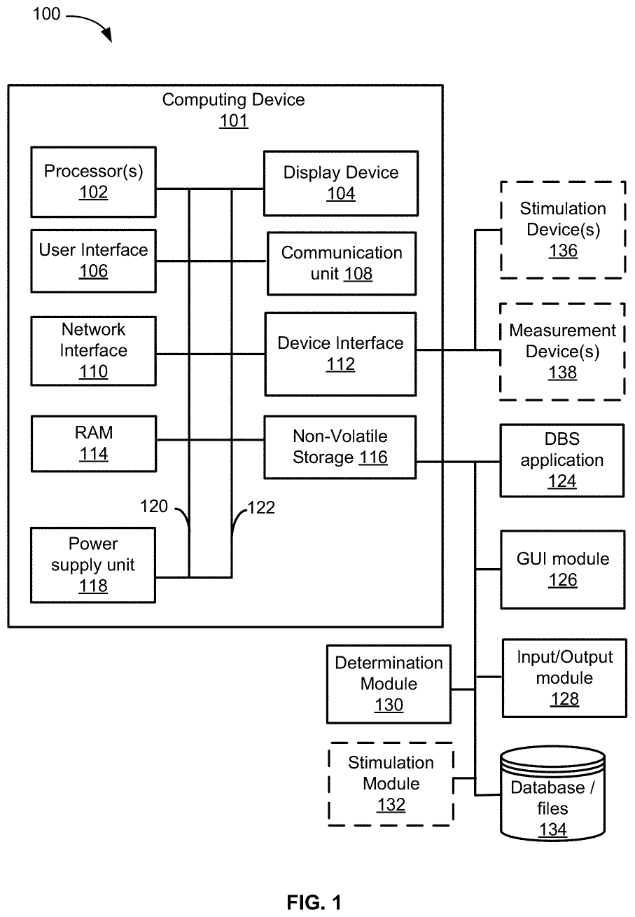

Referring now to FIG. 1, illustrated therein is a diagram of an example embodiment of a system 100 that may be used for predicting neural responses (e.g., excitation/inhibition/suppression) and/or providing stimuli for deep brain stimulation of one or more regions of the brain according to the teachings herein. The system 100 optionally includes components that may be used for delivering deep brain stimulation, according to another example embodiment. In further embodiments, the functions of the system 100 can be run on multiple devices and the functions can be distributed among two or more electronic devices or systems that may be locally or remotely distributed; for example, using cloud-computing resources. FIG. 1 shows various physical and logical components of an example embodiment of the system 100.

As shown, the system 100 includes an electronic device 101 that has a number of physical and logical components, including one or more processors 102, a display device 104, a user interface 106, a communication unit 108, a network interface 110, a device interface 112, a memory unit including random access memory (“RAM”) 114 and non-volatile storage 116, a power supply unit 118 and a communication bus 120 and power bus 122. The communication bus 120 enables the one or more processors 102 to communicate with the other components of the electronic device 101. The various elements of the electronic device 101 can receive power from voltage rails 122 that are provided by the power supply unit 118. The memory 118 may be used to store various programs and data files such as those used to provide an operating system for the electronic device 101 and software modules that are used perform various functions related to DBS such as, but not limited to, a DBS application 124, a GUI module 126, an Input/Output module 128, a determination module 130, and an optional stimulation module 132 as well as database and data files 134. The electronic device 101 may be implemented using a desktop computer, a laptop, a mobile device, a tablet, and the like. In other embodiments the electronic device 101 may include other components and/or have a different configuration while still being able to provide the DBS-related functions discussed herein.

In at least one embodiment, the system 100 may further include stimulation device(s) 136 and/or measurement device(s) 138 in situations where the system 100 is used to apply DBS to a patient and/or perform measurements of electrophysiological signals from the patient.

The one or more processors 102 execute an operating system, and various modules, as described below in greater detail. In embodiments where there are two or more processors, these processors may function in parallel and perform certain functions. The processor(s) 102 control the operation of the device 101 and in some embodiments other components of the system 100. The processor(s) 102 may be any suitable processors, controllers or digital signal processors that can provide sufficient processing power depending on the configuration and operational requirements of the device 101. For example, the processor(s) 102 may include a high-performance processor.

The display device 104 can be any suitable display that provides visual information depending on the configuration of the electronic device 101. For instance, the display device 104 can be a cathode ray tube, a flat screen monitor and the like if the electronic device 101 is a desktop computer. In other cases, the display device 104 can be a display suitable for a laptop, tablet or handheld device such as an LCD-based display and the like. The display device 104 can provide notifications to the user of the electronic device 101. In some cases, the display device 104 may be used to provide one or more GUIs through an Application Programming Interface. A user may then interact with the one or more GUIs for configuring the system 100 to operate in a certain fashion.

The user interface 106 enables an administrator or user to provide input via an input device, which may be, for example, any combination of a mouse, a keyboard, a trackpad, a thumbwheel, a trackball, voice recognition, a touchscreen and the like depending on the particular implementation of the electronic device 101. The user interface 106 also outputs information to output devices, which may be, for example, any combination of the display device 104, a printer or a speaker.

The communication unit 108 is optional and can be a radio that communicates utilizing CDMA, GSM, GPRS or Bluetooth protocol according to standards such as IEEE 802.11a, 802.11b, 802.11g, or 802.11n. The communication unit 108 can provide the processor(s) 102 with a way of communicating wirelessly with certain components of the system 100 or with other devices or computers that are remote from the system 100.

The network interface 110 permits communication with a network, or other electronic devices and/or servers, which may be remotely located from the electronic device 101 but accessible by some network. The network interface 110 may also include other interfaces that allow the electronic device 101 to communicate with other devices or computers. For example, the network interface 110 may include at any combination of an Internet, Local Area Network (LAN), Ethernet, Firewire, modem or digital subscriber line connection.

The device interface 112 can be used to interface with one or more devices; for example, one or more stimulation devices 136 (e.g., a deep brain stimulator) and, in some cases, one or more measurement devices 138, such as sensors like electrodes (e.g., preferably microelectrodes but in some cases macroelectrodes may be used), for example, which may or may not be incorporated with the stimulator device(s) 136. For example, the contacts on clinical DBS leads may be used as macroelectrodes. In some cases, the device interface 112 can include any combination of a serial port, a parallel port or a USB port that provides USB connectivity.

The device interface 112 may also include hardware that allows the processor(s) to send data, such as any combination of control signals, stimulus parameters and/or stimulus waveforms to the stimulation device(s) 136 and to receive measurement data from the measurement device(s) 138. Accordingly, the device interface 112 may include one or more analog to digital converters (ADCs) and one or more digital to analog converters (DACs).

Signal processing hardware may be included in the device interface 112 or as a separate preprocessing unit (not shown) that is coupled to the electronic device 101 in order to pre-process the measurement data. The preprocessing that is done may include standard signal processing techniques such as, but not limited to, any combination of amplification, and/or filtering using parameters that can be determined from experimentation as is known by those skilled in the art.

The RAM 114 and the non-volatile storage 116 may be provided in a memory unit or a data store. The RAM 114 provides relatively responsive volatile storage to the processor(s) 102. The non-volatile storage 116 stores program instructions, including computer-executable instructions, for implementing the operating system and software modules, as well as storing any data used by these software modules. The data may be stored in database or data files 134, such as for data relating to patients that are treated using the system 100. The database/data files 134 can be used to store data for the system 200 such as system settings, parameter values, and calibration data. The database/data files 134 can also store other data required for the operation of the DBS application 124 or the operating system such as dynamically linked libraries and the like. For example, the database/data files 134 can also store data related to the various models that are employed to simulate neuronal excitation in response to certain stimuli parameters that may be provided by a user.

During operation of the system 100, the software instructions for the operating system, and the software modules, as well as any related data may be retrieved from the non-volatile storage 116 and placed in RAM 114 to facilitate more efficient execution. Other computing structures and architectures may be used as appropriate.

The power supply unit 118 can be any suitable power source or power conversion hardware that provides power to the various components of the electronic device 101. The power supply unit 118 may be a power adaptor or a rechargeable battery pack depending on the implementation of the electronic device 101 as is known by those skilled in the art. In some cases, the power supply unit 118 may include a surge protector that is connected to a mains power line and a power converter that is connected to the surge protector (both not shown). The surge protector protects the power supply unit 118 from any voltage or current spikes in the main power line and the power converter converts the power to a lower level that is suitable for use by the various elements of the electronic device 101. In other embodiments, the power supply unit 118 may include other components for providing power or backup power as is known by those skilled in the art.

The stimulation device(s) 136 and sensor(s) 138 provide direct interaction with the brain of a patient. In an example, the stimulation device(s) 140 is a deep brain stimulator that can use pulsatile stimulation with biphasic (thus charge balanced) pulses. The stimulation waveform can be selected such that it enables generation of an appropriate neural response to stimulation (i.e., elicits action potentials that subsequently result in the controlled release of neurotransmitter), with minimal risk of stimulation-induced tissue damage. In some cases, a technician or medical clinician can choose between 30 μs, 60 μs, and 120 μs as pulse widths as appropriate, and adjust the current intensity as appropriate and use the system 100 to deliver the DBS stimuli to the patient. In some cases, the user, such as a medical clinician, researcher or neurosurgeon, can use the electronic device 101 to determine the stimulus parameters that may be used to provide more effective treatment to the patient depending on the condition and/or brain area of the patient that is being treated as discussed in more detail below.

For example, in at least one embodiment, the electronic device 101 further includes a number of software modules/programs with software instructions that are to be executed on the one or more processors 102 for determining DBS stimuli for achieving a desired treatment and/or performing treatment on the patient using the determined DBS stimuli. The software programs that may be executed by the processor(s) 102 include, but are not limited to, the DBS application 124, the GUI module 126, the input/output module 128, the determination module 130, and the optional stimulation module 132. It should be noted that in alternative embodiments, the software instructions may be organized in a different manner, i.e., by a different number of software modules, as long as the functionality described herein is provided.

Advantageously, the electronic device 101 executes the software instructions for at least the DBS application 124 and the determination module to determine one or more stimulus parameters, such as frequency, and generate inhibitory and excitatory synaptic responses (i.e., changes to neuronal currents), and scale these responses with respect to adjusting certain inputs that are relevant for the brain area that is being stimulated such as the proportions of inhibitory and exhibitory inputs. In at least one embodiment, the electronic device 101 can summate these responses to generate a net response to a single stimulus pulse that is used for DBS. After the net response for a single stimulus-evoked response (i.e., the net response) is created, in at least one embodiment, the electronic device 101 can manipulate fidelity of this response based upon a frequency at which successive pulses are delivered (e.g., stimulation frequency-dependence). In general, the neuronal response to the successive pulse stimuli can be different when, for example, 5 Hz is used compared to when 100 Hz is used in the DBS, based on the area of the brain that is receiving the stimuli. These different neuronal responses can be automatically generated (e.g., predicted) based on a model of short-term synaptic plasticity, which manipulates the strength of the synaptic response based on the timing of successive pulses.

The DBS application 124 includes program instructions that when executed by the processor(s) 102 configure the processor(s) 102 to implement one or more methods for allowing the user to perform one or more DBS related functions. For example, the DBS application 124 may instruct the processor(s) to execute the software instructions of the GUI module 126 to provide a user interface to allow the user to interact with the DBS application 124 so that the processor(s) may receive control inputs and various parameter values and other data to allow for any combination of: (a) the generation and/or simulation of DBS stimuli, (b) the output of simulated neural responses to DBS stimuli, (c) the performance of DBS treatment on a patient and/or (d) the measurement of neuronal responses from a patient during DBS treatment. The DBS application 124 may provide such functionality by instructing the processor(s) to execute the software instructions for the input/output module 128, the determination module 130 and/or the stimulation module 132.

When executing the DBS application 124, the processor(s) 102 may also store various operational parameters such as stimulus parameters, patient data, status data, as well as measurement data from neural responses measured from patients, predicted neuronal response data, error data for the differences between the measurement data and the predicted neuronal response data. In at least one embodiment, the processor(s) 102 may be configured to store such data on a remote data store, such as in the cloud for example. In at least one embodiment, alternatively or in addition, the processor(s) may be configured to communicate with other devices to send any portion of the aforementioned data to one or more of these devices. This communication may also include email communication or other forms of electronic messaging.

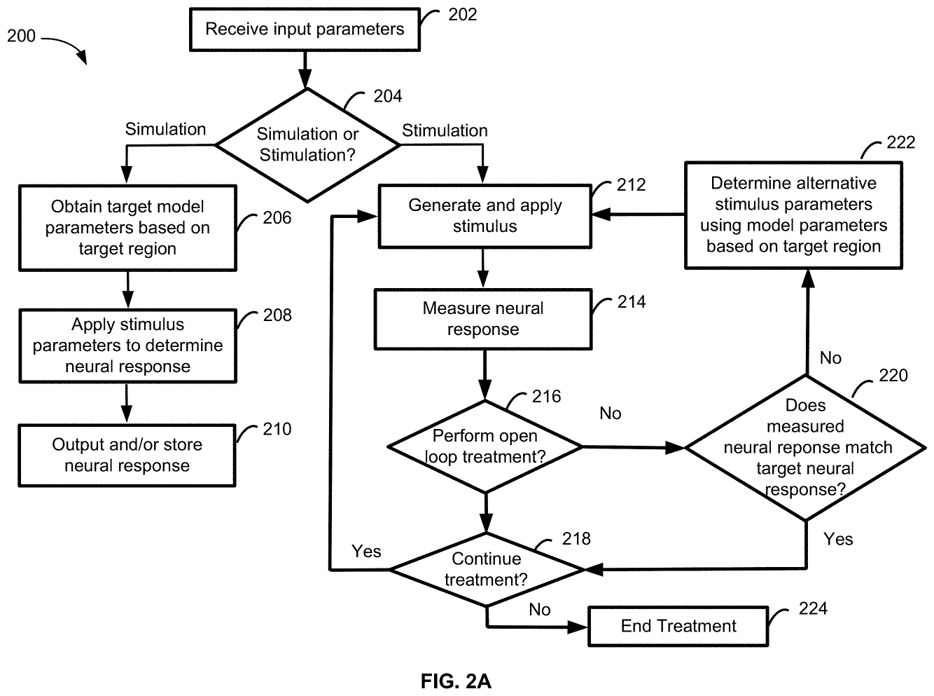

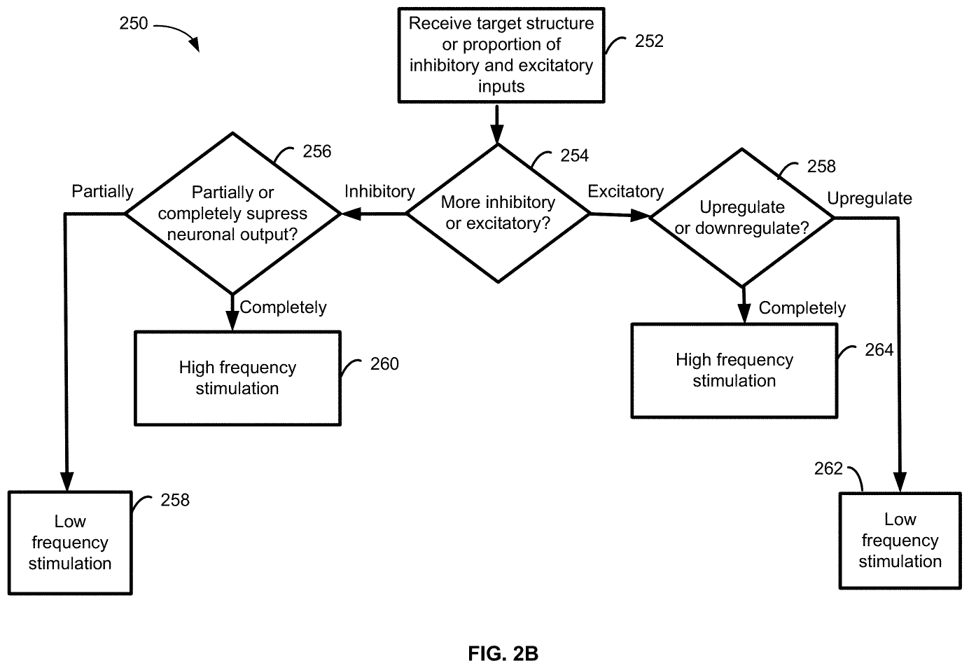

In at least one embodiment, the DBS application 124 may perform method 200 (see FIG. 2A) and have different modes of operation. For example, the DBS application may receive a control input from the user to operate in a simulation mode, or a stimulation mode which includes performing open loop and/or closed loop stimulation. These modes of operation are further discussed with respect to FIG. 2A.

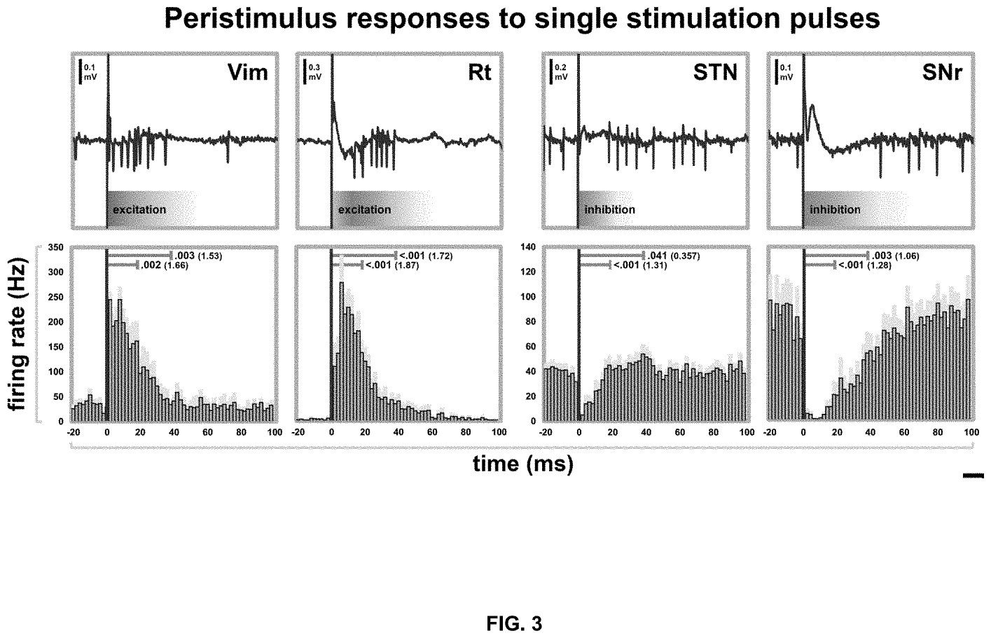

The GUI module 126 includes program instructions that, when executed by the processor(s) 102, configure the processor(s) 102 to generate various GUIs that are then displayed on the display device 104, or another visual output device, to allow the user to perform various functions such as selecting the mode of operation of the DBS application 124 and hence the electronic device 101. The GUI module 126 also includes software instructions for displaying simulation results and/or measurement data depending on the mode of operation of the DBS application 124. Examples of measured and/or simulated neuronal responses that may be provided are similar to those shown in any of FIG. 3, 5A, 5B, or 7 to 12.

The input/output module 128 includes software instructions that, when executed by the processor(s) 102, configure the processor(s) 102 to store data in the databases/files 134 or retrieve data from the databases/files 134. For example, any input data from the user, such as control inputs (e.g., for selecting a mode of operation), stimulus parameters and/or patient data (such as, but not limited to, identity, age, and physiological condition) that is received through one of the GUIs can be stored. In addition, any simulated and/or measured data may be provided from the input/output module 128 to the GUI module 126 for display on the display device 104. Alternatively, or in addition thereto, such simulated and/or measured data may be provided by the input/output module 128 to the communication unit 108 or network interface 110 for transmission to another electronic device and/or remote storage device.

The determination module 130 includes program instructions which may be referred to as determination program instructions. The determination program instructions, when executed by the processor(s) 102, configure the processor(s) 102 to determine neuronal responses for a certain brain region based on inputted stimulus parameters by applying input parameters to a model framework described in accordance with the teachings herein. The determination module 130 may then instruct the processor(s) 102 to perform any combination of: displaying the generated neuronal responses on the display device 104 via the input/output module 128 and the GUI module 126, storing the generated neuronal responses via the input/output module 128 and/or transmitting the neuronal response data to another electronic device via the input/output module 128 as well as the network interface and/or the communication unit 108.

The optional stimulation module 132 can be included in embodiments of the system 100 that include the stimulation device(s) 136 and the measurement device(s) 138. The stimulation module 132 may include program instructions, which may be referred to as stimulation program instructions. The stimulation program instructions, when executed by the processor(s) 102, generates DBS stimuli and provides them to the stimulation device(s) for application to a patient. Any combination of the intensity level (e.g., current and/or voltage amplitude), waveform shape (e.g., pulse width), frequency (e.g., pulse train frequency), number of pulses and overall time duration of the DBS stimuli may be set though the stimulation module 132. The stimulation module 132 may also include program instructions that, when executed by the processor(s) 102, cause measurement data to be obtained from the patient via the measurement device(s) 138.

### Target Model: Framework

In at least one embodiment of the electronic device 101, the target model that is used to predict the effect of DBS pulses on the afferents of the stimulated nuclei for a target structure, such as a region or area of the brain, includes a leaky integrate and fire (LIF) single neuron model, together with a TM model of short-term synaptic plasticity [1]. Each model neuron may be specified to receive a certain number of presynaptic inputs, such as 500 presynaptic inputs, for example, as well as the proportion of those presynaptic inputs which are excitatory and inhibitory, which may be obtained using morphological data (detailed below in “Target Model: Presynaptic inputs”), which may be stored in various libraries or files in the database/files 134 or via input data provided by the user. In general, a different number of presynaptic inputs may be used in other alternatives. However, a pool of 500 neurons was found to be sufficient to provide a biologically realistic model.

In addition to these inputs, in at least one embodiment, the background synaptic activity [2] may be modelled by an Ornstein-Uhlenbeck (OU) process and added to the model neuron to reproduce the impact of synaptic noise that exists in vivo [2,3].

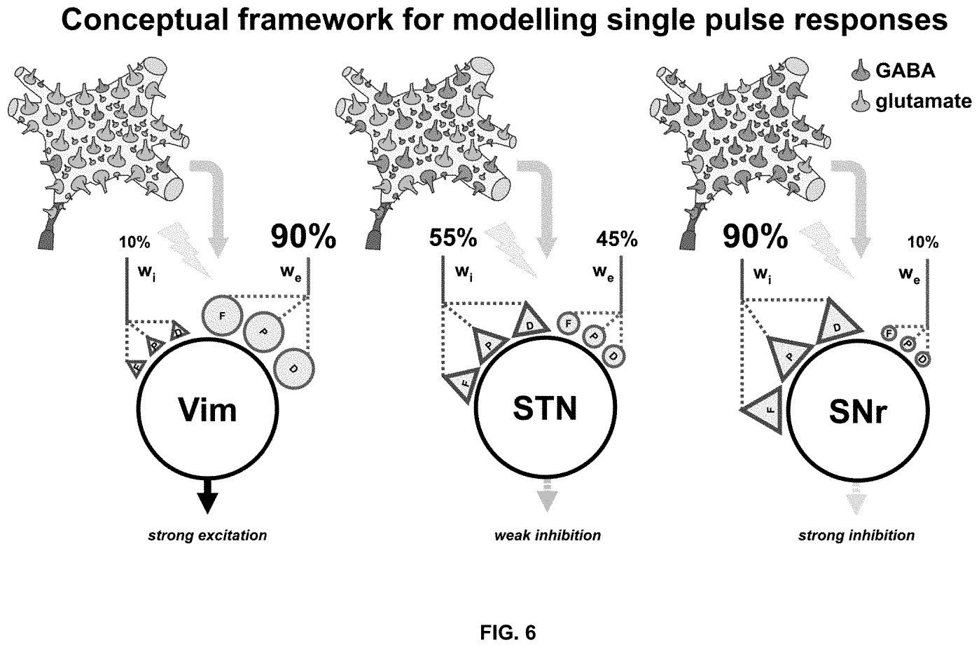

In accordance with the target model framework, each DBS single pulse simultaneously activates all presynaptic inputs (e.g., see FIG. 6). This simultaneous activation was modelled by artificially generating precise spike times which correspond to the arrival of each DBS pulse in the presynaptic inputs. The modeling framework may be used to recreate the neuronal firing in Vim, STN, and SNr in response to stimulation trains with certain frequencies. For example, the computational simulations discussed herein use stimulation trains with frequencies (i.e., pulse rate repetitions) of 1, 2, 5, 10, 20, 30, 50, and 100 Hz, however other frequencies may also be used. In an alternative embodiment, the modeling framework may include other elements such as a new compartment to for more complicated model neurons.

In some cases, model generation for Rt neurons may not be needed to avoid redundancy since the target model parameters are identical to Vim except for the parameters which underly the baseline firing rates (this is further discussed in the “Target Model: Parameter settings” subsection below).

Referring now to FIG. 6, shown therein is an example target model of a response to single pulses of electrical stimulation for several modelled neurons to provide strong excitation, weak inhibition and strong inhibition, respectively, for example. The effect of each DBS single pulse can be modelled by simultaneously activating all presynaptic inputs. To model the response to single pulses of electrical stimulation, each model neuron was assigned a certain proportion of excitatory and inhibitory presynaptic inputs. The proportions may be defined using weights whose values may be derived from anatomical/morphological data as explained below. In FIG. 6, the symbol F means facilitatory, the symbol D means depressive, and the symbol P means pseudolinear and these symbols F are used to indicate the different types of synapses.

### Target Model: Presynaptic Inputs

The vast majority of inputs to the Vim are glutamatergic projections from the dentate nucleus of the cerebellum [4-7] and reciprocal connections from the cerebral cortex [8, 9], with less prominent inputs coming via inhibitory Rt projections [7, 10, 11]. The Rt is a thin sheet of neurons that forms a shell around the lateral and anterior borders of the dorsal thalamus, and to some extent ventral thalamus [12]. It is primarily innervated by collateral branches of glutamatergic thalamocortical and corticothalamic projections [12-16], but also receives less prominent GABAergic innervation from the GPe and SNr [17-19]. Similar to Vim, the majority of afferent inputs to Rt are glutamatergic. The vast majority (˜90%) of inputs to the SNr are GABAergic, projecting from the striatum and globus pallidus externus (GPe) [20, 21], whereas the STN receives a more homogenous convergence of GABAergic and glutamatergic inputs from the GPe [22] and motor cortical areas [23] respectively [20, 24]. While the mixed inputs are more homogenous in STN, electron-microscopy work suggests that GABAergic terminals nevertheless outnumber glutamatergic terminals [25]. Based on the cited literature, estimates of the proportions of inhibitory and excitatory inputs were generated (see Table 6) to be used for the target model framework.

In the target model framework, an ensemble of 500 LIF model neurons, which may be modeled by the LIF model, produced inputs to the stimulated nuclei. Each neuron receives a random input (which may be modelled by using the OU process with a time constant of 5 ms) and fired at the rate of about 5 Hz (the total average firing rate across neurons was equal to 5±0.7 Hz). Each of the 500 neurons was labeled either as excitatory or inhibitory based upon estimates of the proportions of excitatory and inhibitory inputs received by Vim, STN, and SNr (see Table 6). The action potentials (or spikes) of these neurons may be provided to the stimulated nuclei through using a short-term synaptic plasticity model, such as the Tsodyks-Markram (TM) model, for example. The parameters that may be used for the LIF neuron model (see Supplementary Tables 2 and 5 for the LIF parameters) to generate membrane potentials or currents of the stimulated nuclei. The total synaptic current may be obtained as a linear combination of presynaptic excitatory (I_{exc}) and inhibitory currents (6):

I_{syn}(t)=W_{exc}I_{exc}(t)+WᵢₙₕIᵢₙₕ(t) (1)

where W_{exc} and Wᵢₙₕ denote weights of excitatory and inhibitory currents, respectively. These weights, together with the mean and standard deviation (std) of the background synaptic current, can be tuned to produce the neuronal firing rates at the baseline (no DBS) as well as in response to DBS with different frequencies. For example, the tuning may be done using an automated parameter fitting approach. The mean and std of total current may also be determined for each substructure (see TABLE 2).

### Target Model: Synapses

The target model framework can make use of the following equations to model the function of short-term synaptic plasticity according to the Tsodyks-Markram (TM) model:

$$\begin{matrix}\frac{du}{dt} = - \frac{u}{{τ}_{F}} + U ( 1 - {u}^{-} ) δ ( t - {t}_{sp} ) & ( 2 ) \\ \frac{dr}{dt} = \frac{1 - r}{{τ}_{D}} + {u}^{+} {r}^{-} δ ( t - {t}_{sp} ) & ( 3 ) \\ \frac{dI}{dt} = - \frac{I}{{τ}_{s}} + A {u}^{+} {r}^{-} δ ( t - {t}_{sp} ) & ( 4 )\end{matrix}$$

where u indicates utilization probability, i.e., the probability of releasing neurotransmitters in the synaptic cleft due to calcium ion flux in the presynaptic terminal. The current (I) in equation 4 may be used to determine the Iₑₓₜ and Iᵢₙₕ, that are used in equation 1 according to parameters defined in the TABLES shown herein. Upon the arrival of each presynaptic spike, tₛₚ, u increases by U(1−u⁻) and then decays to zero by the facilitation time constant, τ_{f}. The parameter U denotes the increment of u produced by each presynaptic spike and the parameter A denotes the absolute synaptic efficacy of the synaptic connections. The vesicle depletion process, due to the release of neurotransmitters, can be modeled by equation 2, where r denotes the fraction of available resources after neurotransmitter depletion. In contrast to the increase of u upon the arrival of each presynaptic spike, r drops and then recovers, according to the depression time constant τ_{D}, to its steady state value of 1. The competition between the depression (τ_{D}) and facilitation (τ_{f}) time constants determine the dynamics of the synapse. In the TM model, the parameters U, τ_{f}, and τ_{D} are used to determine the type and dynamics of the synapse. In equation 4 used in the TM model, the parameters I and τₛ indicate the presynaptic current and its time constant, respectively. In an example, the time constants of the excitatory and inhibitory inputs may be selected to be about 3 ms and 10 ms, respectively.

### Target Model: Background Synaptic Activity

The target model framework may use the OU process with a time constant of, for example, about 5 ms to represent the effect of synaptic noise. The OU process can be modelled as:

$$\frac{dx}{dt} = - \frac{x ( t ) - μ}{τ} + a \sqrt{\frac{2}{τ}} ξ ( t ) \qquad ( 5 )$$

where ξ is a random number drawn from a Gaussian distribution with 0 average and unit variance, τ is the time constant, μ and α indicate the mean and standard deviation of variable x, respectively. For example, the mean and std dev may be to 0 and 1, respectively, which is the default setting for the OU process. However, since the parameter values in TABLE 2 may be used to scale the total current for each brain region this means that the std dev values in TABLE 2 will be considered for the OU process in an indirect way.

### Target Model: Neurons

The dynamics of the membrane potential in an LIF model can be determined as:

$$\frac{dV ( t )}{dt} = \frac{- ( V ( t ) - {E}_{L} ) + {RI}_{inj} ( t )}{{τ}_{V}} \qquad ( 6 )$$

where, in at least one embodiment, E_{L}=−70 mV, R=1MΩ, and τᵥ=10 ms. The parameter Iᵢₙⱼ indicates the total injected current to the model neuron (i.e., I_{syn} of equation 1 plus the background synaptic noise of equation 5). Furthermore, the current Iᵢₙₕ from equation 6 may be the current determined from equation 1 plus the variable x from equation 5 scaled with certain parameter values obtained from TABLE 2 or in other words the current Iᵢₙₕ=I_{syn}+mean (from Table 2)+std (from Table 2)*x. A spike occurs when V≥Vₜₕ, where Vₜₕ=−40 mV and the reset voltage is −90 mV with an absolute refractory period of 1 msec. These modelling parameters may be determined based on physiological observation.

### Target Model: Parameter Settings

For the target model framework, the inventors determined the proportions of excitatory and inhibitory neurons (see TABLE 1), the total synaptic current (see TABLE 2), the parameters of excitatory synapses (see TABLE 3), the parameters of inhibitory synapses (see TABLE 4), and the time constants of membrane dynamics and synaptic currents (see TABLE 5) that may be used in at least one embodiment described herein. The values in TABLES 3 and 4 were derived from previous experimental work [26] as well as the values in TABLE 5 [27]. The setting of parameter values for TABLES 1 and 2 was determined as described above. The modelling for Rt may be optional due to redundancy as all parameters may be identical to Vim except for the parameters which mediate the baseline firing rates (i.e., W_{exc}, Wᵢₙₕ (but with almost the same ratio) and parameters of background synaptic noise (for TABLE 2). However, the parameters for Rt modelling are included in the TABLES.

TABLE 1
Proportions of excitatory and inhibitory presynaptic inputs

| | number of excitatory | |
|---|---|---|
| | neurons | number of inhibitory neurons |
| STN | 225 (45%) | 275 (55%) |
| SNr | 50 (10%) | 450 (90%) |
| Vim / Rt | 450 (90%) | 50 (10%) |

TABLE 2
Parameters for total current fed to the neuron

| | mean (pA) | st. dev. (pA) | wₑ | wᵢ |
|---|---|---|---|---|
| STN | 32 | 11 | 1.1 | 0.6 |
| SNr | 55 | 10 | 4.5 | 3.3 |
| Vim | 30 | 45 | 25.8 | 65.2 |
| Rt | 12 | 10 | 3.0 | 7.9 |

TABLE 3
Parameters of excitatory synapses

| | facilitation | | | depression | | | pseudo linear | | |
|---|---|---|---|---|---|---|---|---|---|
| | T_{D} | T_{U} | | T_{D} | T_{U} | | T_{D} | T_{U} | |
| | (ms) | (ms) | U | (ms) | (ms) | U | (ms) | (ms) | U |
| STN, | 138 | 670 | 0.09 | 671 | 17 | 0.5 | 329 | 326 | 0.29 |
| SNr, | | | | | | | | | |
| Vim/Rt | | | | | | | | | |

TABLE 4
Parameters of inhibitory synapses

| | facilitation | | | depression | | | pseudo linear | | |
|---|---|---|---|---|---|---|---|---|---|
| | T_{D} | T_{U} | | T_{D} | T_{U} | | T_{D} | T_{U} | |
| | (ms) | (ms) | U | (ms) | (ms) | U | (ms) | (ms) | U |
| STN, | 45 | 376 | 0.016 | 706 | 21 | 0.25 | 144 | 62 | 0.29 |
| SNr, | | | | | | | | | |
| Vim/Rt | | | | | | | | | |

In the target model framework, for inhibitory presynaptic neurons, the ratios of facilitation, depression, and pseudo linear synapses for all three substructures were 0.3, 0.4, and 0.3, respectively. For excitatory presynaptic neurons, the above ratios were used for STN and SNr; however, 0.5, 0.3, and 0.2, respectively, were used for Vim/Rt. Alternatively, it may be possible to use other values in other embodiments.

TABLE 5
Time constants of membrane dynamics and synaptic currents

| | T_{V} (msec) | T_{Exc} (msec) | T_{Inh} (msec) |
|---|---|---|---|
| STN | 12 | 3 | 10 |
| SNr | 10 | 3 | 10 |
| Vim / Rt | 50 | 5 | 8.5 |

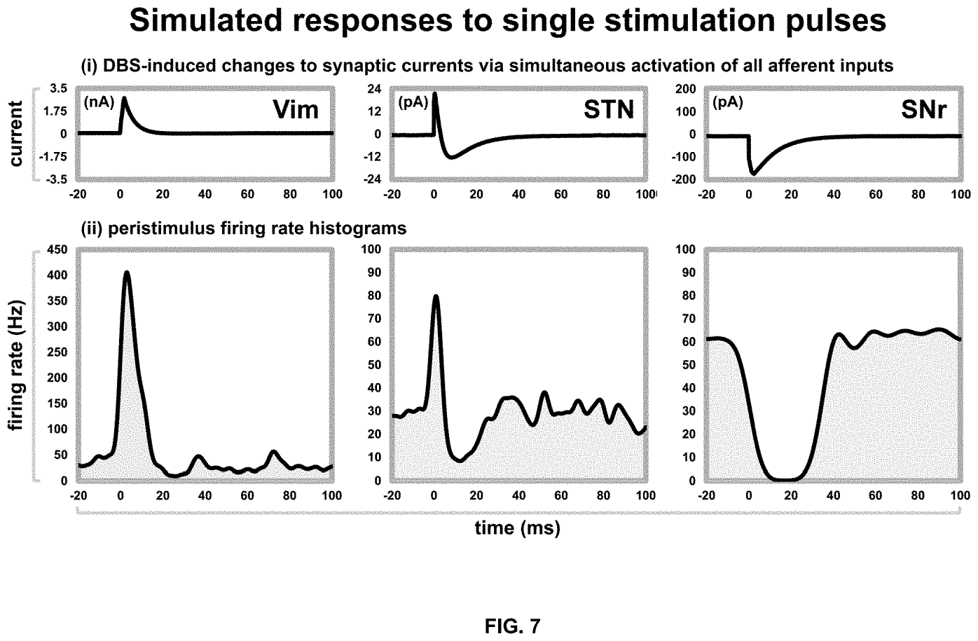

Referring now to FIG. 7, shown therein are examples of corresponding changes to synaptic currents (top panels) and somatic firing (bottom panels) induced by the simultaneous activations (i.e., the single-pulse responses) based on computations that were performed using the target model framework described herein. The results shown in FIG. 7 closely replicated the robust stimulus-evoked neuronal excitation in Vim and neuronal inhibition in SNr in the experimental results (discussed further below). In STN, there was a short-latency neuronal excitation which was not observed in the experimental results (though may have been occluded by the stimulation artifact) due to the high speed of excitatory synaptic transmission, followed by an inhibitory period which is congruent with the experimental data.

### Target Model Output

The various equations described above, and the parameters specified in TABLES 1-5 are used together to generate neuronal output based on user inputs. These neuronal outputs may then be displayed, stored and/or sent to another electronic device.

Referring now to FIG. 2A, shown therein is a flowchart of an example embodiment of a method 200 for predicting neural excitation and/or providing stimuli for deep brain stimulation, in accordance with the teachings herein. The method is performed at least in in part by the processor(s) 102.

At step 202, the method 202 receives user input data which may include the mode of operation (e.g., simulation or stimulation), brain region or target and optionally one or more stimulus parameters for simulating DBS and/or performing DBS treatment.

At step 204 if the operation mode is a simulation mode, then the method 200 proceeds to step 206. Alternatively, if the operation mode is a stimulation mode, then the method 200 proceeds to step 212.

During simulation mode, at step 206, the processor(s) 102 retrieves the target model parameters for the target structure (i.e., brain region) specified by the user. For example, the user may specify one of the SNr, Gpi, Vim or Rt brain regions. The target model parameters may be retrieved from the database/data files 134. Alternatively, the user may specify the target model parameters in the user input which is received by the processor(s) 102. The user input may also specify stimulus parameters such as pulse train frequency and optionally pulse amplitude and/or pulse width. In cases where the user input does not include all of the stimulus parameters, the processor(s) may obtain standard values from the database/data files 134 for the pulse amplitude and/or pulse width. It should be noted that the target model framework described herein may be applied to other structures and is not limited to the SNr, Gpi, Vim or Rt brain regions. For example, in other embodiments, parameters for the target model framework may be for outputs of other neural structures.

At step 208, the processor(s) applies the model parameters and the stimulus parameters to the target model, as described above, to generate the neuronal output (i.e., the neuronal excitation/inhibition/suppression). The application of the model parameters to the target model may be done as described previously.

At step 210, the processor(s) 102 then performs any combination of displaying the neuronal results on the display device 104, storing the neuronal results on the data in the database/files 134 and/or sending the neuronal results to another electronic device.

At this point the user may wish to perform further simulation in which case steps 202, 206, 208 and 210 may be repeated in order to obtain neuronal output that may be more reflective of effective treatment if the DBS stimulus was applied to a patient during a treatment session.

Alternatively, if at step 204 it is determined that the user wishes to perform stimulation, then the method proceeds to step 212. The stimulation may be based on a DBS stimulus that was obtained via simulation as per steps 206 to 210 of method 200. The stimulation may be performed according to an open-loop stimulation mode which involves performing steps 212 to 218 or a closed loop mode which involves performing steps 212 to 222 based on the user input.

With either stimulation mode, the method proceeds to step 212 where the DBS stimulus is generated via the stimulation module 132 and applied to the patient via the stimulation device(s) 136. The stimulus parameters used to generate the DBS stimulus may be specified by the user input. Alternatively, the stimulus parameters may be retrieved from the database/files 134. An example embodiment of a method that may be used at step 212 for applying the DBS stimulus is method 250 which is shown in FIG. 2B.

The method 200 then proceeds to step 214 where the neuronal response from the patient is measured using the measurement device(s) 138. These measurements may be referred to as measurement data, neural measurement data or neuronal measurement data. The measurement data may be displayed on the display device 104, stored in the database/files 134, and/or transmitted to another electronic device.

The method 200 then proceeds to step 216 where it is determined whether the device 101 is operating in open loop stimulation or closed loop stimulation mode. If the device 101 is operating under open loop stimulation mode, then the method 200 proceeds to step 218 where it is determined whether the user wishes to apply another DBS stimulus. If this determination is true, then the method 200 returns to step 212. If the user does not wish to apply another DBS stimulus, then the method 200 proceeds to step 224 and the treatment ends.

Alternatively, if it is determined at step 216 that the device 101 is being operated in closed loop mode, then the method 200 proceeds to step 220 where it is determined whether the measured neuronal output is similar to a desired neuronal output indicating that effective treatment is being performed. This desired neuronal output may be specified by input data that is received from the user. Alternatively, the desired neuronal output may be obtained empirically and stored in the database/files 134 from which it is retrieved during closed loop stimulation. The similarity between the measured neuronal output and the desired neuronal output may be determined by obtaining an error signal from the difference between the measured neuronal output and the desired neuronal output. A measure of the error signal, such as the mean square average error may then be compared to a threshold and if it is larger than the threshold then one or more of the stimulus parameters may be adjusted at step 222 so that the next neuronal response that is generated in response to the next DBS stimulus that is generated and applied at step 212.

Turning to FIG. 2B, a flowchart of a method 250 for delivering deep brain stimulation 200, in accordance with an example embodiment of the teachings herein, is shown.

At step 252, the input/output module 128 is used to receive input data from the user regarding the target structure that will receive the DBS stimulus. The input data may be received from the user via the user interface 106 or the network interface 110. The input data may include the name of the target structure in which case the proportion of inhibitory and exhibitory inputs may be obtained from the database/files 134. Alternatively, the input data may comprise the proportions of inhibitory and exhibitory inputs for a stimulation target structure. In some cases, the proportions of inhibitory and excitatory inputs can be accessed from relevant literature in the art or previous experimental anatomical or histological data and inputted manually by the user and received via the user interface 106 or the network interface 110 along with the input/output module 126.

At step 254, the determination module 116 includes software instructions to configure the processor(s) 102 to compare the number of inhibitory inputs to the number of exhibitory inputs from the input proportions of inhibitory and exhibitory inputs to determine whether there are more inhibitory or exhibitory inputs associated with the target structure.

At step 256, where there are more inhibitory inputs, the determination module 116 includes software instructions to configure the processor(s) 102 to determine whether to partially or completely suppress neuronal output for the target structure. In some cases, such determination can be based on further input data provided by the user and received from the user interface 106 in terms of hypothesis-driven stimulation for the purpose of performing the DBS treatment. This is in contrast to other approaches where the clinician generally blindly inputs a stimulation frequency without knowing what is happening to the target structure. In some cases, the user input can be based on previous knowledge or hypothesis; for example, knowing that the Vim in patients with tremors exhibits pathological tremor-related bursting behaviour, and it may be advantageous to suppress this neuronal output to abolish the pathological activity. In other examples, the hypothesis based on other indications may mean it may be favourable to increase neuronal output, for example.

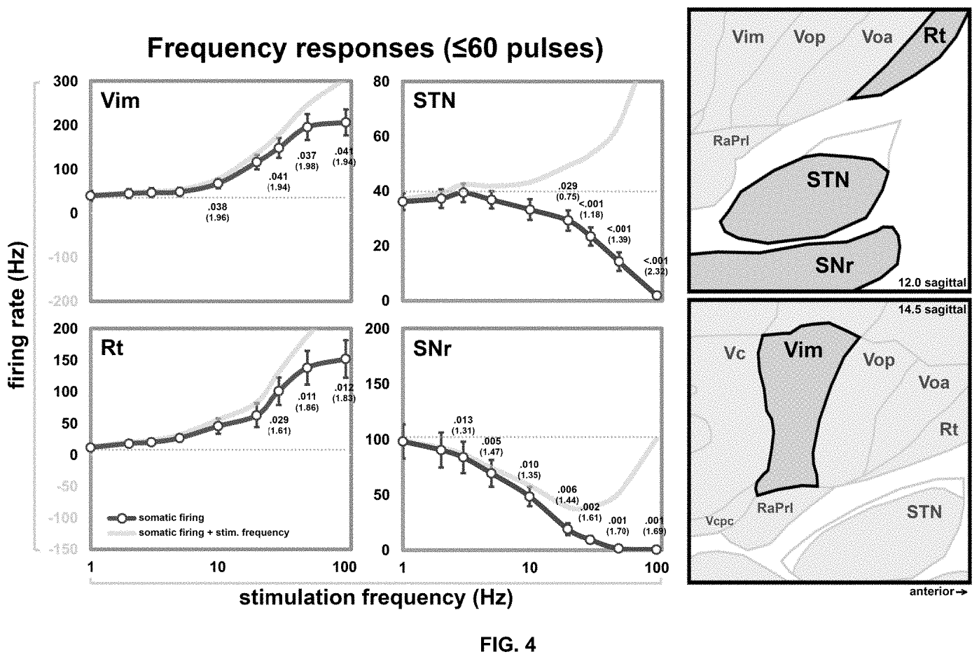

At step 258, where it has been determined to partially suppress neuronal output (e.g., the user may decide based on their understanding of the disease and the brain circuitry of that disease), the stimulation module 132 includes software instructions to configure the processor(s) 102 to direct the stimulation device(s) 136 to deliver a low frequency stimulation such as, for example, less than about 10 to about 20 Hz (e.g. to provide stimulation that does not elicit synaptic depression). In target structures, such as certain regions of the brain, that receive predominantly inhibitory inputs, it is generally not possible to upregulate neuronal activity (as shown in FIG. 1 with respect to STN and SNr, for example). Single pulses of stimulation generally inhibit neuronal activity. If the stimulator device(s) 136 deliver stimulation pulses at a low rate (i.e., a low frequency as described earlier in this paragraph), it can produce periodic inhibitory responses (i.e., partial suppression). If the stimulator device(s) 136 delivers pulses at a fast rate (e.g., a high frequency such as about 100 Hz or more), the inhibitory responses may become “temporally summated”, and thus neuronal output may generally be completely inhibited. FIG. 4 illustrates examples of frequencies for partial versus complete neuronal suppression for different target structures of the brain. In these examples, for STN, complete suppression may be achieved with about 100 Hz stimulation, and for SNr, complete suppression may be achieved with about 50 Hz or greater up to about 200 Hz or so based on the stimulation device(s) 136 that is used.

At step 260, where it has been determined to completely suppress neuronal output by the user, the stimulation module 118 includes software instructions to configure the processor(s) 102 to direct the stimulation device(s) 136 to deliver a high frequency stimulation (e.g., above about 100 Hz). For target structures with predominantly inhibitory inputs (like STN and SNr, for example), the example of FIG. 3 show that each stimulus pulse produces an inhibitory response of a certain duration. Thus, the frequency required to completely suppress neuronal output is equal to 1 divided by the duration of the inhibitory response. For example, when the STN is inhibited for about 10 ms, a frequency of about 100 Hz is used for near complete suppression. As another example, when SNr is inhibited for about 20 ms, a frequency of about 50 Hz is used for near complete suppression.

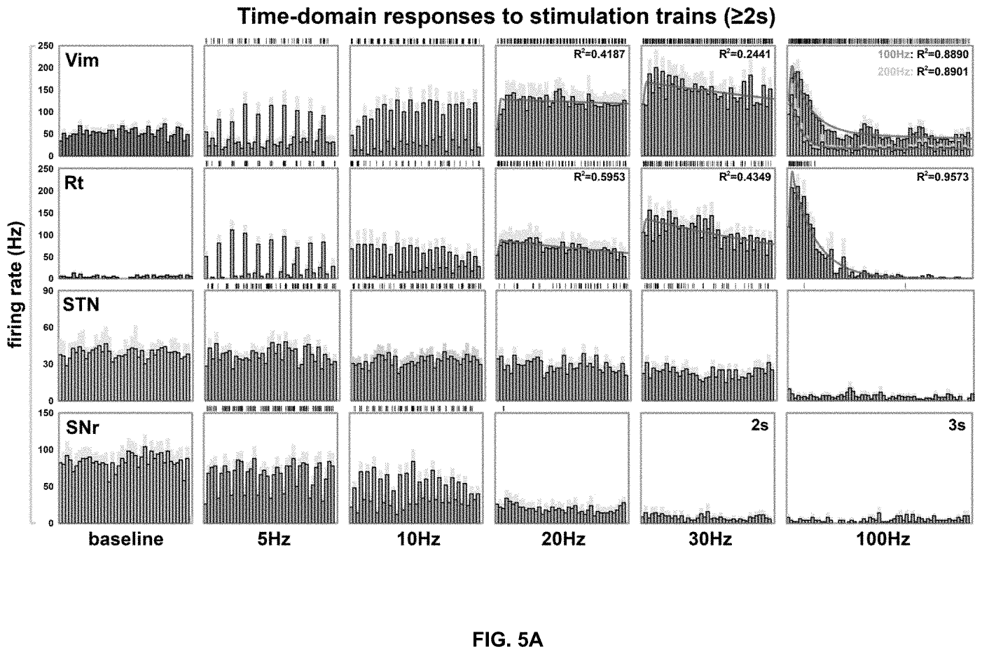

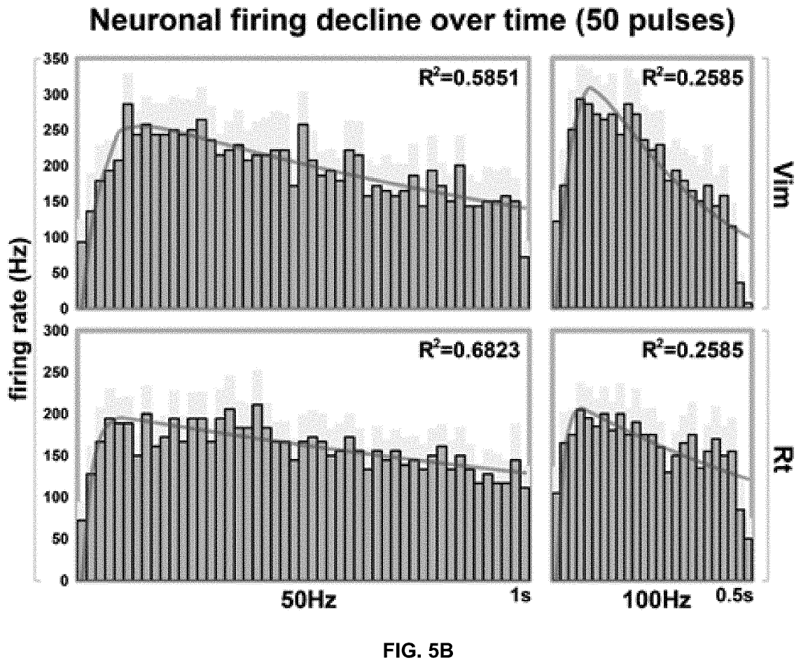

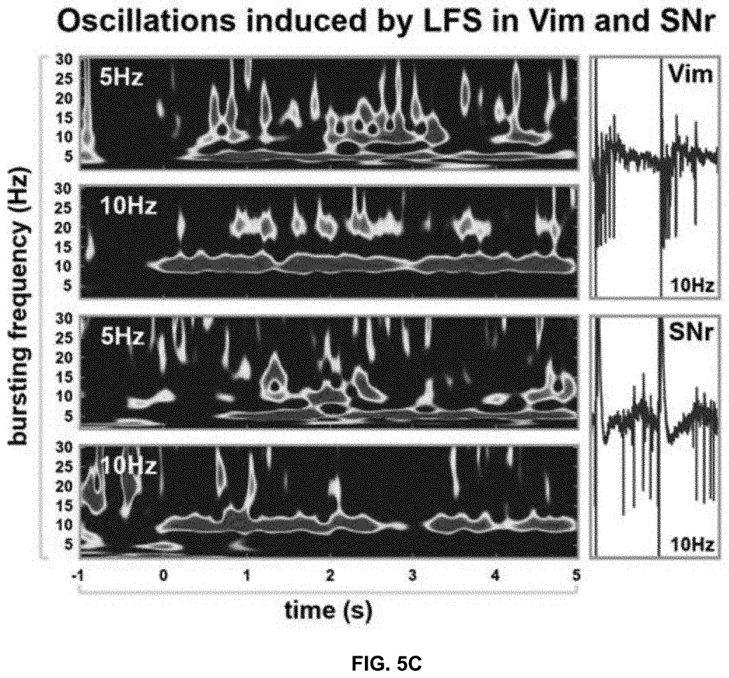

Alternatively, for target structures with predominantly excitatory inputs (like Vim or Rt, for example), each stimulus pulse elicits an excitatory response. When the stimulator device(s) 136 stimulates with higher and higher frequencies such as above about 30 Hz for example, there is temporal summation of excitatory neuronal responses, thus upregulating activity more and more. In this way, short-term synaptic plasticity becomes important. When stimulation is delivered at, for example, 30 Hz, for Vim or Rt each successive stimulation pulse produces a weaker and weaker response. For example, for Vim or Rt, with stimulus frequencies of about 100 Hz, or more, there is occlusion of neurotransmitter release due to how fast the stimuli are being delivered; thus, glutamate (an excitatory neurotransmitter) is likely not being released beyond an initial first few seconds (as illustrated in the example of FIG. 5 with respect to Vim and Rt at stimulation frequencies of about 100 Hz and about 200 Hz).

At step 258, where there are more excitatory inputs than inhibitory inputs for the specified target structure, the determination module 116 includes software instructions to configure the processor(s) 102 to determine whether to upregulate or downregulate neuronal activity by providing appropriate control signals to the stimulation device(s) 136. In some cases, such determination can be based on user input received from the user interface 106. Accordingly, the electronic device 101 allows a user/clinician to make an informed decision regarding what effect is desirable to incur onto brain activity. In some cases, the informed decision can be based on previous knowledge or hypothesis; for example, wanting to improve this patient's memory, and surmising that it might be beneficial to upregulate the output of the hippocampus, which may be done, for example, by reducing the output of the STN or GPi in Parkinson's disease.

At step 262, where it has been determined to upregulate neuronal activity, the stimulation module 118 includes software instructions to configure the processor(s) 102 to direct the stimulation device(s) 136 to deliver a low frequency stimulation, which will differ depending on the target structure and may be determined from empirical evidence. For example, low frequency stimulation in Vim and Rt generally elicits neurotransmitter release reliably, thus inducing action potential firing. At step 264, where it has been determined to downregulate neuronal activity, the stimulation module 118 includes software instructions to configure the processor(s) 102 to direct the stimulation device 136 to deliver a high frequency stimulation, which may be about 100 Hz or higher. As an example, using stimulation of 100 Hz and greater may instead induce synaptic depression and occlude neurotransmitter release, thus suppressing “synaptic communication” and not eliciting the excitatory responses that are achievable with low frequencies that do not elicit neurotransmitter depression (but instead reliably activate these synapses). As an example, frequencies under 100 Hz generally upregulates neuronal activity. In this example, for structures like the Vim and Rt, frequencies at about 100 Hz and above generally begin to suppress neuronal output.

The various embodiments described herein model certain neurological characteristics of the brain, which may be determined from the general population, and provide bespoke stimulation based on the region of the brain that is being stimulated. For example, in regions of the brain that have predominantly inhibitory inputs (e.g., SNr or GPi), one cannot selectively activate glutamatergic inputs and ignore the (predominant) GABAergic inputs, and thus one cannot make the neurons fire. However, fortunately in a region with predominantly excitatory inputs, the target model described herein can be used to provide stimulation to produce action potential firing. In addition, the target model can be used to suppress neuronal output by stimulating at a sufficiently high frequency, which occludes neurotransmitter release entirely, thus blocking synaptic communication and reducing the neuronal output.

The target model described herein takes into consideration anatomical and physiological properties to predict the brain-region-specific and/or frequency-dependent effects of deep brain stimulation on neuronal activity in a target structure in the human brain. In order to make such predictions, the target model incorporates anatomical properties, in part, by accounting for the inhibitory/excitatory proportions in inputs to the target structure, which may come from any suitable source, such as anatomical/histological literature, to predict an expected response to single pulses of stimulation. In another aspect, the target model described herein accounts for physiological properties by considering short-term synaptic dynamics such as, for example, frequency-dependent effects on synaptic depression. For example, one can activate the presynaptic terminals to an upper frequency limit at which point there is insufficient time for the input terminals to the neurons of the target structure to recover and to be able to reliably release neurotransmitters. This can be accounted for by application of a model to account for short-term plasticity such as, but not limited to, the Tsodyks-Markram model.

The target model described herein allows for brain-region-specificity by modelling certain anatomical properties of the stimulated brain region to determine a net response to a single pulse of electrical stimulation. In this way, a weighted composition of afferent inputs (i.e., proportions of inhibitory inputs versus excitatory inputs) may be used to determine whether a stimulus pulse will produce an excitatory response (upregulate neuronal firing), an inhibitory response (downregulate neuronal firing), or a net-neutral response.

Frequency-dependency means that when stimuli are delivered successively with DBS, the target model incorporates certain physiological properties of the stimulated brain region to determine the strength of the neuronal response to each stimulus pulse based upon the duration of time between consecutive pulses in the stimulus. In general, with greater rates or frequencies of stimulation pulses, synaptic transmission fidelity is reduced. Thus, for example, in a region of the brain in which a single stimulus pulse (or a low frequency train of stimulation pulses) elicit excitatory effects (i.e., upregulate neuronal output), higher stimulation frequencies suppress synaptic transmission and downregulate neuronal output.

By taking into account brain-region-specificity and frequency-dependency, the target model described herein may be used to provide information on whether stimulation in a particular brain region (i.e., target structure), at a particular frequency, may result in upregulation or downregulation of neuronal activity at the particular brain region. Thus, the target model described herein can make be used to provide physiologically informed stimulation in a functional manner (i.e., knowledgably increasing or decreasing communication across brain regions). In other words, a clinician may make a decision whether it might be most favourable to suppress elicit neuronal firing for a given disease in a given target structure and use the target model framework to determine stimuli to suppress elicit neuronal firing for the given target structure.

### Experimental and Computational Study

In order to demonstrate how effective the systems and methods described herein are at predicting the effects of certain stimuli at certain locations in the brain, a computational study based on at least one embodiment of the systems and methods described herein was performed and compared to an experimental study that was performed. In the experimental study microelectrode recordings of single-neuron activity across four brain regions (ventral intermedius nucleus (Vim), thalamic reticular nucleus (Rt), subthalamic nucleus (STN), and substantia nigra pars reticulata (SNr)) during stimulation trains across a range of frequencies (1-100 Hz) were obtained. The methodology and results of the experimental and computational studies are now discussed.

### Methods

Experimental study: Patients and Neurons

The study that was performed included 115 neurons from patients with Parkinson's disease (n=47) or essential tremor (n=11). All experiments conformed to the guidelines set by the Tri-Council Policy on Ethical Conduct for Research Involving Humans and were approved by the University Health Network Research Ethics Board. Moreover, each patient provided written informed consent prior to taking part in the studies.

### Experimental Study: Protocols

Neurophysiological mapping procedures were performed during awake DBS surgeries (OFF-medication) using two closely spaced microelectrodes (600 μm apart, 0.1-0.4 MΩ impedances) [29]. Techniques for identification of Rt, STN, SNr [30], and Vim [28, 31] neurons have been previously reported. One microelectrode was used for recording single-neuron activity while a second immediately adjacent microelectrode was used to deliver stimulation trains at different frequencies. Recordings were obtained using two Guideline System GS3000 amplifiers (Axon Instruments, Union City, Calif.) and signals were digitized at >=12.5 kHz with a CED 1401 data acquisition system (Cambridge Electronic Design, Cambridge, UK). Microstimulation was delivered using an isolated constant-current stimulator (Neuro-Amp1A, Axon Instruments, Union City, Calif.) with 0.3 ms biphasic pulses (cathodal followed by anodal).

To generate stimulation frequency response functions, stimulation trains were delivered at 1 Hz (10 pulses), 2 Hz (20 pulses), 3 Hz (60 pulses), 5 Hz (50 pulses), 10 Hz (50 pulses), 20 Hz (60 pulses), 30 Hz (60 pulses), 50 Hz (50 pulses), and 100 Hz (50 pulses) using 100 μA and a 0.3 ms biphasic pulse width. This frequency response protocol was executed at 9 Vim (nₚₐₜᵢₑₙₜₛ=5), 11 Rt (nₚₐₜᵢₑₙₜₛ=11), 27 STN (nₚₐₜᵢₑₙₜₛ=16), and 14 SNr (nₚₐₜᵢₑₙₜₛ=9) recording sites. Data for STN and SNr were previously collected [32], whereas Vim and Rt data for this study were unique. Longer trains (>2 s) of 100 Hz stimulation were also delivered to the aforementioned Vim, Rt, and SNr neurons. 100 Hz long train data for STN (44 neurons, nₚₐₜᵢₑₙₜₛ=20) were previously collected [33], as were a subset of 100 Hz and 200 Hz Vim (10 recording sites, nₚₐₜᵢₑₙₜₛ=8) data [28]. The average firing rates of all structures for all frequencies were determined. A summary of the data is shown in Table 6.

TABLE 6
Experimental data summary

| | Vim | Rt | STN | SNr |
|---|---|---|---|---|
| FIG. 1 | Vim1-9 | Rt1-11 | STN1-27 | SNr1-14 |
| | [de novo] | [de novo] | [32] | [32] |
| | (n = 9) | (n = 11) | (n = 27) | (n = 14) |
| FIG. 2 | Vim1-9 | Rt1-11 | STN1-27 | SNr1-14 |
| 5-30 Hz | [de novo] | [de novo] | [32] | [32] |
| | (n = 9) | (n = 11) | (n = 27) | (n = 14) |
| FIG. 2 | Vim10-19 | Rt1-11 | STN28-71 | SNr1, 3-7, |
| 100 Hz | [28] | [de novo] | [33] | 10-13 |
| | (n = 10) | (n = 11) | (n = 44) | (previously |
| | | | | collected; |
| | | | | unpublished; |
| | | | | n = 10) |
| FIG. 5B | Vim1-9 | Rt1-11 | — | — |
| | [de novo] | [de novo] | | |
| | (n = 9) | (n = 11) | | |

### Experimental Study: Offline Analyses and Statistics

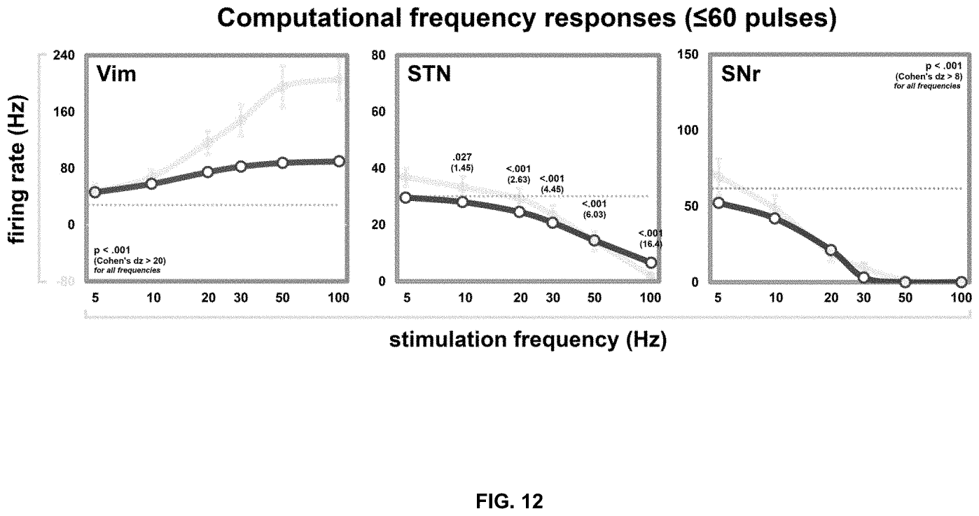

For artifact removal, data from the start of each stimulation artifact to just after the anodic peak (i.e. from the anodic peak or last saturated value to about 25% of the baseline amplitude) were replaced by a straight line; corresponding to a time window of ˜0.8 ms. Data were then high pass filtered (>=250 Hz) and template matching was done using a principal component analysis method in Spike2 (Cambridge Electronic Design, UK). Artifact subtraction allowed for data to be high-pass filtered without distortion in the time domain as otherwise may occur when filtering a signal containing saturated high-amplitude stimulation artifacts [34]. As a single action potential is >=1 ms, then at most one action potential might be lost in the <1 ms artifact subtraction process. With a 0.8 ms artifact removal window, the percentage of data lost during each stimulation train corresponds to: 0.08% (1 Hz), 0.16% (2 Hz); 0.24% (3 Hz); 0.4% (5 Hz); 0.8% (10 Hz); 1.6% (20 Hz); 2.4% (30 Hz); 4% (50 Hz); and 8% (100 Hz). To investigate single-pulses responses, peristimulus histograms (120 ms total width, 20 ms offset, 2 ms bins) were created to encompass responses to all 50 stimuli delivered during the 5 Hz train, across all neurons. The 20 ms pre-stimulus periods were compared to the 20 ms and 40 ms post-stimulus periods using Bonferroni-corrected (two comparisons) two-tailed paired t-tests, and effect sizes (Cohen's d_{z}) were calculated. For the frequency response protocol (<=60 stimulation pulses delivered at each frequency), firing rates were measured before and during each of the stimulation trains. Kolmogorov-Smirnov tests were used to assess the null hypothesis that the data are normally distributed. One-way repeated measures ANOVA tests (stimulation frequency as a within-subject factor) were carried out, and if significant main effects were found, Bonferroni-corrected (nine comparisons) post-hoc t-tests were used to compare firing rates during the various stimulation trains to pre-stimulation baseline firing. ANOVA effect sizes (η²) and t-test effect sizes (Cohen's d_{z}) were also determined. One neuron from the Vim group and one neuron from the Rt group were excluded from statistical analyses due to incomplete stimulation protocol (i.e. missing data points). Of note, the solid gray lines in FIG. 2 consider that each stimulation pulse generated one action potential on the efferent axon [35], representing a situation in which the overall “neuronal output” is the summation of the somatic firing rate and a stimulus-locked efferent axon activation. However, it should be noted that the statistical analyses only consider the action potential firing during periods of time that were not populated by artifacts (i.e. the activity generated at the somatic level). ANOVA analyses were carried out in the same way for both experimental (FIG. 2) and computational (FIG. 12) results. To investigate possible time-varying responses throughout the stimulation trains, time-series histograms (2-3 s total width, no offset, 50 ms bins) were created for 5 Hz, 10 Hz, 20 Hz, 30 Hz, and long trains of 100 Hz (and 200 Hz long trains for Vim). Of note, the long train (i.e. 3 s) 100 Hz (and 200 Hz) data come from various sources since long trains of high-frequency stimulation were not initially delivered (please refer to Table 1 for data summary). The attenuations of excitation over time in Vim and Rt during stimulation trains of >=20 Hz were fit with double exponential functions. Histograms were also created for the shorter trains (<=1 s) of stimulation at 50 Hz and 100 Hz (0.5-1 s total width, no offset, 20 ms bins; FIG. 5B). Moreover, to investigate the prominent time-vary effects in Vim and Rt during 3 s, 100 Hz and 200 Hz stimulation trains, baseline firing was compared to the first second of stimulation and the subsequent 2 s of stimulation using Bonferroni-corrected (two comparisons) two-tailed paired t-tests.

### Experimental Study Results: Responses to Single Stimulation Pulses & Stimulation Frequency Response Functions

Referring now to FIG. 3, the top panels show examples of responses to a single stimulation pulse in each structure, whereas the bottom panels show groupwise firing rate (mean+standard error) peristimulus time histograms of stimulus-evoked excitatory responses for Vim (n=9) and Rt (n=11) and stimulus-evoked inhibitory responses for STN (n=27) and SNr (n=14). The average±standard error baseline firing rates for Vim, Rt, STN, and SNr neurons were 32.0±11 Hz, 8.2±1 Hz, 39.9±3 Hz, and 102.3±16 Hz, respectively. The responses to single stimulation pulses shown in FIG. 3 revealed stimulus-evoked excitatory responses for Vim and Rt, and inhibitory responses for STN and SNr. For Vim, the average firing rates of the immediate 20 ms (181.0 Hz±33 Hz; p=0.002) and 40 ms (125.2±25 Hz; p=0.003) periods following stimulation pulses were significantly greater than the 20 ms pre-stimulus period. This was also the case for the 20 ms (186.2±29 Hz; p<0.001) and 40 ms (120.8±20 Hz; p<0.001) post-stimulus periods for Rt. For STN, the average firing rates of the 20 ms (22.4±3 Hz; p<0.0001) and 40 ms (32.6±4 Hz; p=0.041) post-stimulus periods were significantly less than the 20 ms pre-stimulus period. This was also the case for the 20 ms (7.8±3 Hz; p<0.0001) and 40 ms (21.9±7 Hz; p=0.003) post-stimulus periods for SNr. All statistics were corrected for multiple comparisons. The p-values of Bonferonni-corrected 2-tailed paired t-test are displayed with Cohen's d_{z} effect sizes in parentheses in FIG. 3.

Referring now to FIG. 4, stimulation (<=60 pulses) frequency response functions show that average firing rates progressively increased in Vim and Rt as the stimulation frequency became greater indicating excitatory responses for Vim and Rt, while they progressively decreased in STN and SNr indicating inhibitory responses. The average±standard error baseline firing rates for Vim, Rt, STN, and SNr neurons were 32.0±11 Hz, 8.2±1 Hz, 39.9±3 Hz, and 102.3±16 Hz, respectively (dashed gray lines). Firing rates during the various stimulation trains were compared to the baseline firing rates and the p-values of Bonferroni-corrected post-hoc t-tests (2-tailed, paired) are displayed with Cohen's d_{z} effect sizes in parentheses. The ANOVA main effects for stimulation were all significant. If one considers that each DBS pulse generates an action potential on the efferent axon, then the overall neuronal output may be the summation of the somatic firing rate and stimulation frequency; this is represented by the solid gray lines in each plot (the values on this line for 100 Hz in Vim, Rt, and STN are 100 (Hz) plus the value on the corresponding coloured line). The right anatomical panels are 12.0 mm and 14.5 mm sagittal sections (FIG. 3B shows the locations of the highlighted structures relative to other neuroanatomical landmarks).

In particular, for Vim, neuronal firing rates progressively increased as the stimulation frequency became greater and a significant main effect of stimulation was found [F=43.074 (9,234), p<0.001, η²=0.624]. The Bonferroni-corrected t-tests revealed differences in neuronal firing compared to baseline at stimulation frequencies of 10 Hz (p=0.038), at 30 Hz (p=0.041) and greater (p<0.05). For Rt, neuronal firing rates also progressively increased as the stimulation frequency became greater and a significant main effect of stimulation was found [F=31.170 (9,117), p<0.001, η²=0.706]. Statistically significant differences in neuronal firing compared to baseline were found at stimulation frequencies of 30 Hz (p=0.029) and greater (p<0.05). For STN, neuronal firing rates were progressively attenuated as the stimulation frequency became greater and a significant main effect of stimulation was found [F=26.420 (9,91), p<0.001, η²=0.746]. Statistically significant differences in neuronal firing compared to baseline were found at stimulation frequencies of 20 Hz (p=0.029) and greater (p<0.001). For SNr, neuronal firing rates also progressively attenuated as the stimulation frequency became greater and a significant main effect of stimulation was found [F=25.890 (9,63), p<0.001, η²=0.787]. Statistically significant differences were found at stimulation frequencies of 3 Hz (p<0.05) and greater (p≤0.01). Detailed post-hoc t-test statistics (all corrected for multiple comparisons within the text and figures) and Cohen's d_{z} effect sizes are depicted in FIG. 4.

### Experimental Study Results: Time-Domain Responses to Stimulation

Referring now to FIG. 5A, experimental time-domain responses to stimulation trains are shown. For Vim and Rt, firing rates (mean+standard error) progressively increased with increasing stimulation frequencies. For Vim and Rt, periodic excitatory responses are shown at 5 Hz and 10 Hz, however the strength of the excitatory responses declined overtime with stimulation trains of >=20 Hz and were modelled by double exponential decay functions (R² values within FIG. 5A). The time-series histograms for long train >=100 Hz data (3 s) in Vim and Rt show particularly prominent time-varying responses. In particular, excitatory responses with 100 Hz long trains (>=2 s) were transient, and a subsequent reduction of neuronal firing is evident after the initial excitation. Accordingly, these stimulations elicited excitatory responses that were transient in nature and limited to the start of stimulation.

In Vim, the initial excitatory response at 200 Hz is of shorter duration than at 100 Hz, and the subsequent neuronal suppressive response is stronger at 200 Hz compared to 100 Hz. For example for Vim at 100 Hz (3 s), the firing rate at baseline (41.1±7 Hz) was different from the firing rate during the first 1 s of stimulation (94.8±7 Hz; p=0.004), but not for the subsequent 2 s of stimulation (45.4±7 Hz). However, for Vim at 200 Hz (3 s), the firing rate at baseline (53.1±8 Hz) was not different from the first 1 s of stimulation (35.9±9 Hz; FIG. 5A depicts a very transient initial excitation followed by suppression) but was for the subsequent 2 s (14.0±5 Hz; p=0.002).

For Rt at 100 Hz (3 s), the firing rate at baseline (7.7±1 Hz) was different from the first 1 s of stimulation (90.7±14 Hz; p=0.002), but not for the subsequent 2 s (4.3±2 Hz).

In STN and SNr, there was an overall stationary neuronal suppressive effect with increasing frequency (rather than an effect which changed dynamically over time as was the case in Vim and Rt). In SNr, periodic inhibitory responses are visible at 5 and 10 Hz. Example firing rate raster data from each structure during the various stimulation trains are displayed above each of the panels. This figure is intended to demonstrate the dynamics of the firing rate as a function of time. Of note, the data in FIG. 5A for 5-30 Hz stimulation is the same as that presented in FIG. 4, while the 100 Hz (and 200 Hz) data in FIG. 5A come from various sources since long trains of high-frequency stimulation were not initially delivered (please refer to Table 6 for the data summary).

### Target Model Computational Results: Responses to Single Stimulation Pulses

The net changes to postsynaptic currents in response to single pulses of stimulation may be modelled by simultaneous activations of all presynaptic inputs (see top panels in FIG. 7). These responses differed across brain regions due to differences in the proportions of excitatory and inhibitory inputs (as discussed in the section “Target Model: Presynaptic inputs” and in TABLE 1. The simulated peristimulus firing rate histograms (i.e. the neuronal responses to the aforementioned changes to presynaptic currents) revealed stimulus-evoked excitatory responses for Vim (peak firing rate of 405.9 Hz vs. 245.1 Hz in the experimental data), inhibitory responses for SNr (minimum firing rate of 0 Hz vs. 0.7 Hz in the experimental data), and short-latency excitatory responses (78.8 Hz peak vs. no peak in the experimental data) followed by a longer latency inhibitory response (8.4 Hz trough vs. 4.8 Hz in the experimental data) for STN. The lack of short-latency excitation in the experimental data may be explained by discrepancies in temporal dynamics of excitatory transmission and/or occlusion of this response by the stimulus artifact.

### Target Model Computational Results: Time-Domain Synaptic Currents

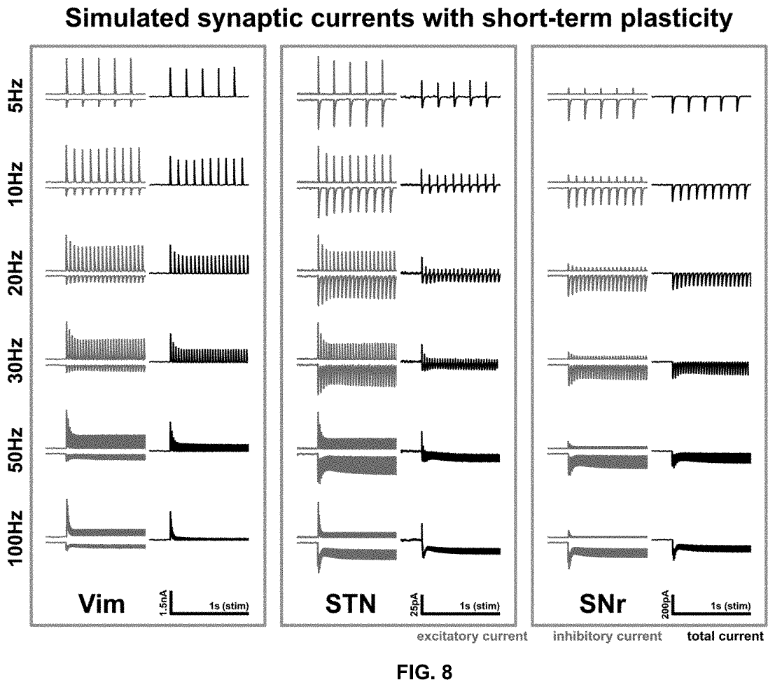

In the computations performed using the target model framework, excitatory and inhibitory synaptic currents were generated separately, along with the total (i.e., sum of excitatory and inhibitory) synaptic currents in responses to DBS pulses across a range of frequencies for each of Vim, STN, and SNr (as illustrated in FIG. 8). The TM model was used to account for frequency-dependent changes to short-term synaptic dynamics. In all structures, the target model suggests frequency-dependent depression of both excitatory and inhibitory synaptic currents. For Vim, sustained (stable) periodic excitations are seen with 5 Hz and 10 Hz, while frequency-dependent weakening of the excitatory responses with successive stimuli are observed with frequencies 20 Hz which corroborates the experimental data. Predominant inhibitory synaptic currents corroborate the strong inhibitions of somatic firing in SNr with low stimulation frequencies, which may be about 20 Hz or lower, whereas neuronal suppression with higher frequencies is likely the result of frequency-dependent synaptic depression. For STN, the mixed excitatory-inhibitory stimulus-evoked responses likely explain the more net-neutral somatic firing responses in experimental data with lower stimulation frequencies, whereas synaptic depression can explain the frequency-dependent suppression of somatic firing with higher stimulation frequencies.

### Target Model Computational Results: Time-Domain Membrane Potentials

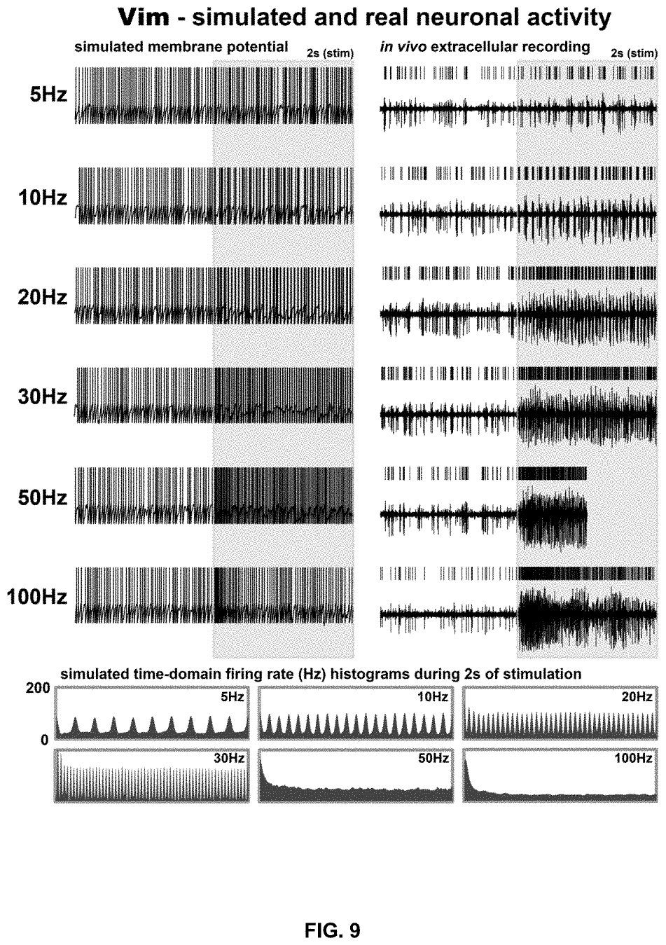

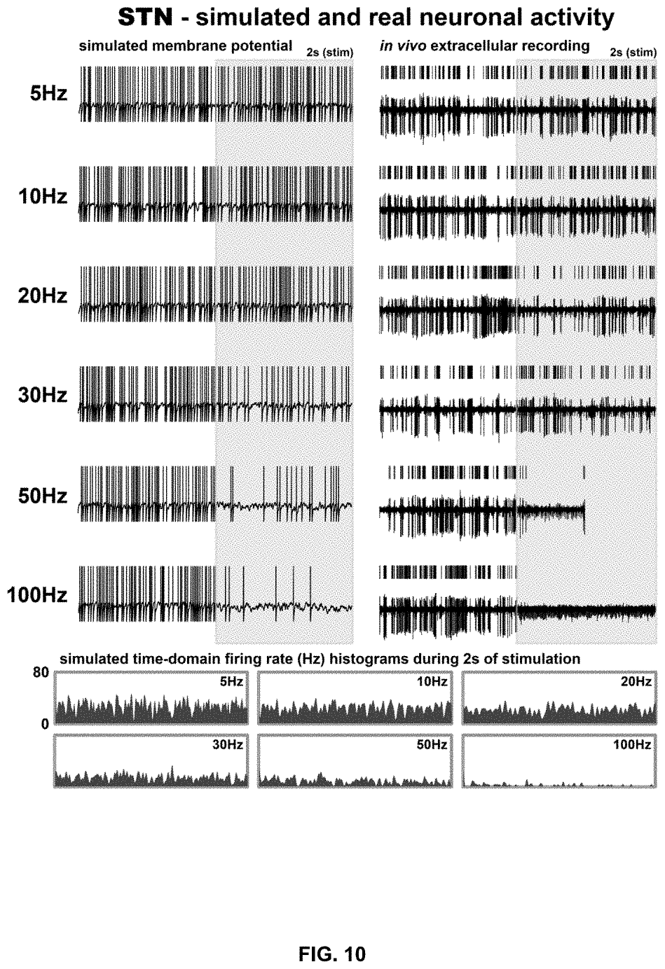

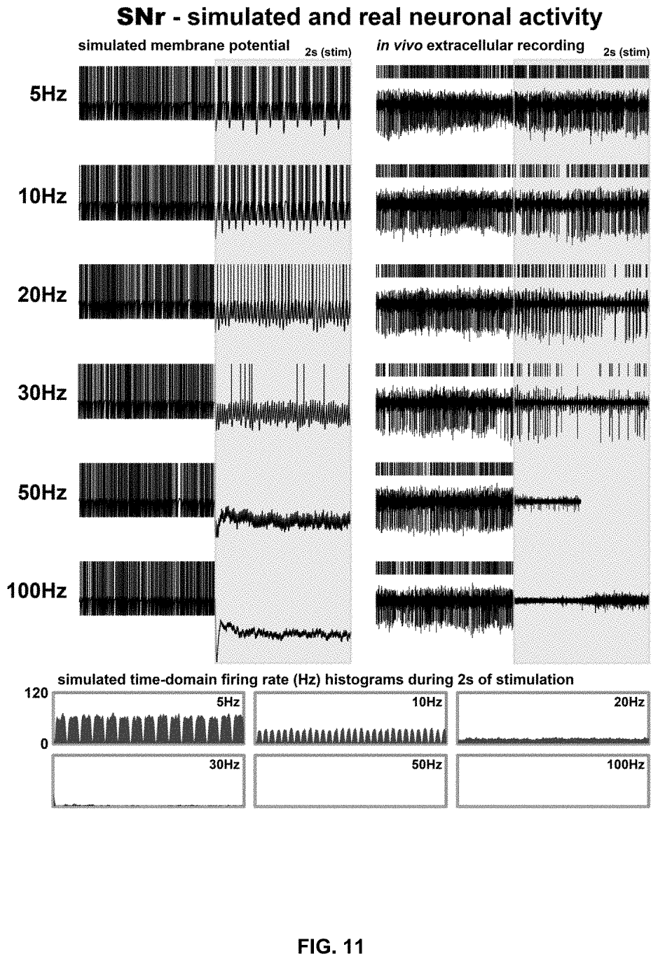

To validate the target model framework, the membrane potentials of modelled neurons in response to DBS, across a range of frequencies, were generated for each of Vim (as illustrated in FIG. 9), STN (as illustrated in FIG. 10), and SNr (as illustrated in FIG. 11). The proportions of excitatory and inhibitory inputs, as illustrated in TABLE 1, together with the parameters of the model neurons, as illustrated in TABLE 2, generated baseline (DBS-OFF) firing rates which corresponded to in vivo recordings. The time-domain histograms that are shown in FIGS. 9, 10, and 11 were generated by averaging the neuronal firing rates of 10 modelled neurons for each respective structure across 2 s of stimulation at each frequency. Any other number of neurons can be modeled and different amounts of stimulation may be applied in the simulation.

For example, referring to FIG. 9, shown therein is computational time-domain membrane potentials for Vim and real voltages (in vivo extracellular recordings) for Vim (predominantly excitatory inputs) in responses to stimulation at different frequencies. The left side panels in FIG. 9 show the simulated membrane potential (accounting also for action potential generation) immediately before (non-shaded) and during (shaded) DBS across a range of frequencies for Vim, whereas the right-side panels show exemplary recordings from an in vivo human Vim neuron (stimulation for 50 Hz was limited to 1 s). The bottom-most panels show time-domain firing rate histograms generated by averaging across 10 model Vim neurons. Synchronous/periodic neuronal firing due to stimulus entrainment is reproduced by the model neuron for DBS at 20 Hz. The model neuron can moreover partially reproduce the transient excitatory responses at DBS onset with 50 Hz and 100 Hz stimulation and 30 Hz to some degree. However, the transient excitatory responses within the modelled neuron are of shorter latency.

Referring now to FIG. 10, shown therein are computationally derived time-domain membrane potentials for STN and real voltages (in vivo extracellular recordings) for STN (mixed inputs) in responses to stimulation at different frequencies. The left panels show the membrane potential of a model STN neuron immediately before (non-shaded) and during (shaded) DBS across a range of frequencies. The right panels are exemplary recordings from an in vivo human STN neuron (stimulation for 50 Hz was limited to 1 s). The bottommost panels show time-domain firing rate histograms generated by averaging across 10 model STN neurons. The simulated (left) neuronal firing rate compared to baseline decreases for DBS at 30 Hz, corroborating experimental data (exemplary in vivo STN neuron portrayed on the right side). The modelled neuronal firing rates are substantially attenuated with DBS at 50 Hz and 100 Hz (as was the case in the experimental results) due to synaptic depression.

Referring now to FIG. 11, shown therein are computationally derived time-domain membrane potentials for SNr and real voltages (in vivo extracellular recordings) for SNr (predominantly inhibitory inputs) in response to stimulation at different frequencies. The left panels show the membrane potential of a model SNr neuron immediately before (non-shaded) and during (shaded) DBS across a range of frequencies. The right panels are exemplary recordings from an in vivo human SNr neuron (stimulation for 50 Hz was limited to 1 s). The bottommost panels are time-domain firing rate histograms generated by averaging across 10 model SNr neurons. The simulated (left panels) neuronal firing rate decreases dramatically beginning at 20 Hz due to the dominant inhibitory presynaptic currents, corroborating experimental data (e.g., the exemplary in vivo SNr neuron portrayed on the right-side panels). The model neuron fails to generate action potentials for DBS 50 Hz (as is the case in the experimental results) due to non-selective synaptic depression.

Computational results: Stimulation frequency response functions

Referring now to FIG. 12, shown therein are computational frequency response functions for Vim, STN and SNr for firing rate versus stimulus frequency. Firing rates during the various stimulation trains were compared to the baseline firing rates and the p-values of Bonferroni-corrected post-hoc t-tests (2-tailed, paired) are displayed with Cohen's d_{z} effect sizes in parentheses. Similar to experimental results, significant main effects of stimulation were found for Vim [F=2400.280 (6, 54), p<0.001, η²=0.996], STN [F=227.963 (6, 54), p<0.001, η²=0.962], and SNr [F=7093.439 (6, 54), p<0.001, η²=0.999]. 10 modelled neurons were used for each brain structure and the stimulation duration at each frequency was constrained to match experimental data within FIG. 2. The post-hoc t-test statistics (corrected for multiple comparisons) and Cohen's d_{z} effect sizes are depicted in FIG. 12. The neuronal dynamics for stimulation (60 pulses) frequency response functions were found to match experimental results (solid gray lines). The average±standard error baseline firing rates for computational Vim, STN, and SNr neurons were 28.0±0.1 Hz, 30.1±0.2 Hz, and 61.7±0.3 Hz, respectively (dashed gray lines). Accordingly, the neuronal dynamics overall matched experimental results, and better results may be obtained by performing further tuning to optimize initial excitatory responses of Vim more precisely.

### Discussion of Computational Results

### Site-Specific and Frequency-Dependent Stimulation Effects

At the somatic level, electrical stimulation from DBS is generally both site-specific and frequency dependent. For example, in Vim and Rt, neuronal activity can be upregulated, whereas in STN and SNr, it can be downregulated depending on the stimulus parameters that are used for DBS provided to these structures. These mechanistic disparities across brain regions are most likely explained by anatomical differences in local microcircuitries, in that the effects appeared dependent upon the weighted composition of excitatory and inhibitory inputs that converge at target neurons. Advantageously, the findings of the experiments described herein illustrate that neuronal activity in any brain region may be suppressed, either selectively in regions with a high predominance of inhibitory inputs, or non-selectively in any brain region when high enough stimulation frequencies were used. Neuronal excitation can be achieved when electrical stimulation is delivered to brain regions with a high predominance of glutamatergic inputs (i.e., excitatory inputs). While these bimodal effects (excitatory vs. inhibitory) with low stimulation frequencies may be attributable to presynaptic activation, the loss of site-specificity and convergence towards neuronal suppression with sustained HFS (100 Hz) may be attributable to synaptic depression. This phenomenon of short-term synaptic plasticity can be defined as a reversible decrease in synaptic efficacy, caused by (i) the depletion of readily releasable neurotransmitter vesicle pools when successive stimuli are delivered at a fast rate, (ii) a reduction of presynaptic calcium conductance, and/or (iii) the inactivation of neurotransmitter release sites due to delayed recovery from vesicle fusion events.

The target model framework described herein was created by the inventors based on the determination that the post-synaptic responses (i.e., neuronal output) to single pulses of electrical stimulation were mediated by the proportions of inhibitory versus excitatory inputs to the stimulated neuron, and that weakened synaptic transmission fidelity over time with higher stimulation frequencies, such as about 100 Hz and greater, was mediated by short-term synaptic plasticity (namely, synaptic depression). As such, in at least one embodiment described herein, the target model framework is a biophysical model framework that is used advantageously takes into consideration both anatomical (local microcircuitry) and physiological (short-term synaptic dynamics) properties. At stimulation frequencies below the threshold for synaptic depression (i.e., <20-30 Hz), the model framework showed that neuronal responses were the result of a temporal summation of stimulus-evoked responses. In structures with predominantly excitatory inputs, this advantageously leads to increases in neuronal output, whereas the opposite occurs in structures with predominantly inhibitory inputs. Beyond the threshold for synaptic depression, in some cases, the strengths of successive stimulus-evoked responses may be progressively reduced such that there may be a loss of synaptic transmission fidelity. In the Vim, with high frequencies, an excitatory response is seen that weakens over time. For example, with frequencies of about 30 Hz and less than 100 Hz the excitatory response weakens over time and with frequencies of about 100 Hz or more the excitatory response drops to or below baseline. In the SNr, stimulus-evoked inhibitory responses were of sufficient magnitude to induce a substantial amount of neuronal inhibition. However, the SNr may also be affected by synaptic depression where there are progressive, frequency-dependent decreases to the amplitudes of extracellular evoked filed potentials in SNr with stimulation frequencies ≥20 Hz. While one may assume that since synaptic depression may weaken the strength of inhibitory synaptic transmission, neuronal firing may increase by disinhibition. However, the target model framework shows non-selective synaptic depression of both inhibitory and excitatory synaptic currents, which corroborates experimental work in rodent STN slices where pharmacologically isolated excitatory and inhibitory postsynaptic potentials were both depressed during HFS [36]. Hence, high-frequency DBS can be considered a “functional deafferentation” [37]. This may also explain the suppression of somatic firing in STN with higher stimulation frequencies of about 50 to 100 Hz or greater, whereas the stimulus-evoked responses with lower frequencies (e.g., less than about 50 Hz for example) may produce rather weak net-neutral inhibitory responses due to the more homogenous distribution of excitatory and inhibitory inputs in STN compared to SNr.

In the example target model framework described herein, both excitatory and inhibitory postsynaptic currents are modelled to generate a site-specific (i.e., dependent upon the proportion of convergent inhibitory/excitatory inputs) and frequency-dependent DBS-mediated net current elicited by each stimulation pulse. Thus, this target model framework may capture stimulation mediated neuronal dynamics across various brain targets and applied stimulation frequencies. Notably, each of the studies that are described herein suggest that short-term synaptic depression may be a putative mechanism of high-frequency DBS. If each DBS pulse generates an axonal action potential [38], then the overall “neuronal output” may be considered as the summation of the somatic firing (that is influenced by afferent axon/axon terminal activations [r16]) plus the direct efferent axonal activations (this summation has been incorporated within FIG. 2). Thus, HFS applications which completely suppresses somatic firing may replace neuronal output with a more regular pattern of output corresponding to the stimulation frequency. However, in cases where somatic firing is not completely suppressed, such as in the STN at lower stimulation frequencies or when stimulating structures such as the Vim and Rt, the effect may be a “summation” of axonal and somatic firing, rather than an explicit decoupling.

### Translational Implications

The selectively bimodal and frequency-dependent somatic responses described herein may be taken into consideration in the development of novel stimulation paradigms and DBS indications. In applications of DBS which utilize a high stimulation frequency, such as about 100 Hz or more, suppression of somatic output is likely achieved for therapeutic purposes (though as mentioned above, axonal activations may also be considered). If each DBS pulse generates an axonal action potential, then the overall neuronal output rate can be considered as the summation of the somatic firing rate and stimulation frequency as shown in FIG. 4. Thus, HFS applications which completely suppresses somatic firing may replace neuronal output with regular outputs corresponding to the stimulation frequency. Stimulation paradigms which utilize low stimulation frequencies and are applied to areas of the brain with predominantly glutamatergic inputs may depend upon periodic facilitation of somatic firing, with one possible example being low-frequency pedunculopontine-DBS [65]. For example, from experimental PPN-DBS trials in the past, 10 Hz and 20 Hz appeared most favourable. Low-frequency stimulation in an area of the brain with predominantly inhibitory inputs may, on the other hand, cause periodic inhibitions. In either case, low-frequency stimulation can induce oscillatory neuronal behaviour (as seen in FIG. 5C). From FIG. 5C, it can be seen that in Vim, oscillatory bursting activity was induced (i.e., entrainment) with low-frequency stimulation by way of periodic stimulus-evoked neuronal excitations. FIG. 5C also shows that in SNr, oscillatory bursting activity was also induced; however, it was caused by periodic stimulus-evoked neuronal inhibitory responses of the otherwise high spontaneous firing rates.

In some cases, knowledge of the site-specific and frequency-dependent properties of DBS can inform the development of novel stimulation paradigms such as closed-loop stimulation for on demand upregulation or downregulation of neuronal firing, as described herein, or for induction or disruption of neuronal oscillations. While stimulation parameters are conventionally often decided upon empirically, based on the teachings here, knowledge of the local microcircuitry (e.g., distribution of afferent inputs) inherent to the stimulated brain region (e.g., therapeutic targets of interest for DBS application) may allow for the inference/prediction of the stimulation frequency response properties. Accordingly, the target model framework described herein may be used as a tool for physiologically informed stimulation programming and paradigm development in prospective DBS targets and indications, particularly as the target model framework was developed based on in vivo experimental data from the human brain.

### Computational Study: Stimulation of the Basal Ganglia

For the stimulation of the basal ganglia, based on the target model framework, it was found that individual stimulation pulses elicited net inhibitory responses in both STN and SNr. These responses are corroborated by anatomical studies which suggest a predominance of GABAergic inputs to each of these structures. The greater predominance of inhibitory inputs to SNr likely explains the stronger inhibitory responses compared to STN. While single pulses of stimulation at low frequencies, such as about 20 Hz or less, elicited inhibitory responses, with higher stimulation frequencies (i.e., 20 Hz up to the limit of the stimulation device(s) 136), neuronal inhibition/suppression was even more easily achieved likely due to a combined effect of inhibitory temporal summation and non-selective synaptic depression.

The canonical model of Parkinson's disease suggests that the activity of the STN is pathologically increased as a result of a lack of downregulation of D2-dopamine-receptor-mediated “indirect-pathway” projections [39-42]. The subsequent increased excitatory drive of subthalamo-pallidal and nigral projections, in combination with a lack of upregulation of D1-dopamine-receptor-mediated “direct-pathway” projections, leads to a pathological overactivity of the GPi and SNr. As such, in Parkinson's disease, the STN, SNr, and GPi benefits from the suppression of neuronal overactivity, and/or stimulation-induced abolishment of pathophysiological oscillations which manifest throughout the disease process.

### Computational Study: Simulation of the Vim

In the Vim, individual stimulation pulses elicited time-locked excitatory responses. While stimulus-evoked inhibitory responses in SNr and STN were likely caused by the predominance of inhibitory afferent inputs, the excitatory responses elicited In Vim are most likely due to the predominant excitatory afferent inputs. In high-frequency stimulation of the Vim, somatic firing was transiently upregulated at the start of stimulation, but this effect weakened progressively over time. The target model framework described herein demonstrates that initial excitatory response may likely be due to synaptic activation of the predominant glutamatergic afferents, while the subsequent weakening may likely be due to synaptic depression due to the fast rate at which subsequent stimuli are delivered. For example, in an intracellular sensorimotor thalamic rat brain slice, it was shown that 125 Hz stimulation resulted in an initial depolarization response coupled with increased neuronal firing, followed quickly by (complete or incomplete) repolarization coupled with suppression of neuronal firing [37]. These responses were reversed by application of glutamate receptor and calcium channel antagonists, demonstrating their presynaptic nature. The neuronal suppression which occurred after the initial excitation was the result of synaptic depression.

The pathology of essential tremor is generally not well understood, but evidence is increasingly indicating a dysfunction and possible degeneration of the cerebellum [43, 44]; which subsequently projects to the Vim. Postmortem studies have revealed pathologies including but not limited to Purkinje cell loss [45, 46], Purkinje cell axonal and dendritic swelling [47, 48], and reduced cerebellar GABAergic tone [49]. These pathological changes, namely GABAergic dysfunction, may drive the characteristic neurophysiological changes that occur in patients with essential tremor [50], including tremor-related oscillations at the local field potential [51] and single-unit levels [52] which are coherent with tremor at the periphery. As such, the therapeutic efficacy of high-frequency, such as about 100 Hz and greater, Vim-DBS can be explained by suppression of neuronal firing and/or abolishment of pathophysiological tremor-related oscillatory activity [28]. Conversely, low-frequency stimulation-induced tremor [53, 54] might be explained by periodic excitations which mimic tremor-related neuronal activity.

### Computational Study: Simulation of Rt

In the Rt, like in the Vim, individual stimulation pulses elicited time-locked excitatory responses. Also, like in the Vim, the upregulation of neuronal firing during stimulation at 20 Hz weakened over time, and with 100 Hz stimulation, the excitatory responses were limited to the first ˜0.5 s of the stimulation train, after which somatic firing was reduced to or below baseline. An in vivo nonhuman primate study [55] demonstrated that inhibitory responses which occurred after 0.5 s trains of microstimulation at 100 Hz (note: neuronal firing was not assessed during stimulation), hypothesized to have been mediated by GABAergic afferent inputs to the Rt. The computational studies discussed herein showed that 0.5 s 100 Hz stimulation initially trains drove neuronal firing during stimulation, which likely reflects the prominent glutamatergic input, after which neuronal firing was suppressed likely due to synaptic depression. Slice work has demonstrated that electrical stimulation of the Rt can elicit EPSCs [56] and neuronal spiking [57, 58]. Moreover, stimulation of layer VI cortical neurons [59], and ventrobasal [60] and ventrolateral [61] thalamic neurons elicited excitatory responses in Rt.

In animal studies, Rt stimulation has been demonstrated as a possible treatment for various forms of epilepsy [62-66]. Hypersynchronization of Rt output has been implicated in spike-wave seizures [67], supporting a rationale for Rt-DBS for intractable epilepsy [68]. Moreover, the GABAergic output of the Rt has an important role in controlling excitability throughout the thalamus, as it is known to project to many different thalamic nuclei [69-72]. In Rt-DBS, upregulation of Rt neuronal activity may have a widespread inhibitory effect on downstream thalamic structures, while suppression of Rt activity may lead to disinhibition (unless its output is replaced by regularized DBS pulses). DBS of other nodes of the ascending reticular activating system have also been investigated for minimally conscious state [73, 74] due to the role of this system in behavioural arousal and consciousness. Moreover, stimulation of the pontomesencephalic tegmentum area has been shown to modulate sleep in patients with Parkinson's disease [75, 76]. Considering the role of Rt in arousal and attention [77, 78] and the role of synchronous neuronal activity (which can be induced by stimulation as shown in FIG. 5C) during sleep [58], stimulation of the Rt may be useful in the treatment of sleep disorders.

At least one embodiment of the target model framework described herein can be used to generate stimuli for certain regions of the brain that take advantage of the site-specific and frequency-dependent nature of DBS at the somatic level. Neuronal suppression may be achieved either by stimulus-evoked inhibitory events in structures with predominantly GABAergic inputs (e.g., for the STN and SNr) or non-selectively when sustained HFS was delivered. Stimulus-evoked neuronal excitatory responses were seen in structures with predominantly glutamatergic inputs (e.g., for the Vim and Rt), particularly with lower stimulation frequencies. The bimodal site-specific stimulus-evoked responses may be due to differences in the weighted composition of inhibitory and excitatory inputs to the stimulated target structures, whereas convergence towards neuronal suppression with sustained HFS may be due to synaptic depression.

The embodiments of the present disclosure described above are intended to be examples only and it is not intended that the applicant's teachings be limited to such embodiments. The present disclosure may be embodied in other specific forms. Alterations, modifications, and variations to the disclosure may be made without departing from the intended scope of the present disclosure. While the systems, devices, and processes disclosed and shown herein may comprise a specific number of elements/components, the systems, devices, and assemblies may be modified to include additional or fewer of such elements/components. For example, while any of the elements/components disclosed may be referenced as being singular, the embodiments disclosed herein may be modified to include a plurality of such elements/components. Selected features from one or more of the example embodiments described herein in accordance with the teachings herein may be combined to create alternative embodiments that are not explicitly described. All values and sub-ranges within disclosed ranges are also disclosed. The subject matter described herein intends to cover and embrace all suitable changes in technology. The entire disclosures of all references recited above are incorporated herein by reference.

### REFERENCES

1. Tsodyks M, Pawelzik K, Markram H. Neural networks with dynamic synapses. Neural Comput 1998; 10:821-35. https://doi.org/10.1162/089976698300017502.
2. Destexhe A, Rudolph M, Fellous J-M, Sejnowski T J. Fluctuating synaptic conductances recreate in vivo-like activity in neocortical neurons. Neuroscience 2001; 107:13-24. https://doi.org/10.1016/S0306-4522(01)00344-X.
3. Destexhe A, Rudolph M, Par e D. The high-conductance state of neocortical neurons in vivo. Nat Rev Neurosci 2003; 4:739-51. https://doi.org/10.1038/nrn1198.
4. Asanuma C, Thach W T, Jones E G. Distribution of cerebellar terminations and their relation to other afferent terminations in the ventral lateral thalamic region of the monkey. Brain Res Rev 1983; 5:237-65. https://doi.org/10.1016/0165-0173(83)90015-2.
5. Kultas-Ilinsky K, Ilinsky I A. Fine structure of the ventral lateral nucleus (VL) of the Macaca mulatta thalamus: cell types and synaptology. J Comp Neurol 1991; 314:319-49. https://doi.org/10.1002/cne.903140209.
6. Ilinsky I A, Kultas-Ilinsky K. Motor thalamic circuits in primates with emphasis on the area targeted in treatment of movement disorders. Mov Disord 2002; 17:S9-14. https://doi.org/10.1002/mds.10137.
7. Kuramoto E, Fujiyama F, Nakamura K C, Tanaka Y, Hioki H, Kaneko T. Complementary distribution of glutamatergic cerebellar and GABAergic basal ganglia afferents to the rat motor thalamic nuclei. Eur J Neurosci 2011; 33:95-109. https://doi.org/10.1111/j.1460-9568.2010.07481.x.
8. Stepniewska I, Preuss T M, Kaas J H. Thalamic connections of the primary motor cortex (M1) of owl monkeys. J Comp Neurol 1994; 349:558-82. https://doi.org/10.1002/cne.903490405.
9. Kakei S, Na J, Shinoda Y. Thalamic terminal morphology and distribution of single corticothalamic axons originating from layers 5 and 6 of the cat motor cortex. J Comp Neurol 2001; 437:170-85. https://doi.org/10.1002/cne.1277.
10. Ambardekar A V, Ilinsky I A, Froestl W, Bowery N G, Kultas-Ilinsky K. Distribution and properties of GABAB antagonist [3H]CGP 62349 binding in the rhesus monkey thalamus and basal ganglia and the influence of lesions in the reticular thalamic nucleus. Neuroscience 1999; 93:1339-47. https://doi.org/10.1016/S0306-4522(99)00282-1.
11. Ilinsky I A, Ambardekar A V, Kultas-Ilinsky K. Organization of projections from the anterior pole of the nucleus reticularis thalami (NRT) to subdivisions of the motor thalamus: light and electron microscopic studies in the Rhesus monkey. J Comp Neurol 1999; 409:369-84. https://doi.org/10.1002/(SICI) 1096-9861(19990705)409:3<369::AID-CNE3>3.0.CO;2-H.
12. Jones E G. Some aspects of the organization of the thalamic reticular complex. J Comp Neurol 1975; 162:285-308. https://doi.org/10.1002/cne.901620302.
13. Crabtree J W. The somatotopic organization within the cat's thalamic reticular nucleus. Eur J Neurosci 1992; 4:1352-61. https://doi.org/10.1111/j.1460-9568.1992.tb00160.x.
14. Crabtree J W. The somatotopic organization within the rabbit's thalamic reticular nucleus. Eur J Neurosci 1992; 4:1343-51. https://doi.org/10.1111/j.1460-9568.1992.tb00159.x.
15. Gonzalo-Ruiz A, Lieberman A R. GABAergic projections from the thalamic reticular nucleus to the anteroventral and anterodorsal thalamic nuclei of the rat. J Chem Neuroanat 1995; 9:165-74. https://doi.org/10.1016/0891-0618(95)00078-X.
16. Pinault D. The thalamic reticular nucleus: structure, function and concept. Brain Res Rev 2004; 46:1-31. https://doi.org/10.1016/j.brainresrev.2004.04.008.
17. Par e D, Hazrati L-N, Parent A, Steriade M. Substantia nigra pars reticulata projects to the reticular thalamic nucleus of the cat: a morphological and electrophysiological study. Brain Res 1990; 535:139-46. https://doi.org/10.1016/0006-8993(90)91832-2.
18. Hazrati L-N, Parent A. Projection from the external pallidum to the reticular thalamic nucleus in the squirrel monkey. Brain Res 1991; 550:142-6. https://doi.org/10.1016/0006-8993(91)90418-U.
19. Asanuma C. GABAergic and pallidal terminals in the thalamic reticular nucleus of squirrel monkeys. Exp Brain Res 1994; 101:439-51. https://doi.org/10.1007/BF00227337.
20. Parent A, Hazrati L-N. Functional anatomy of the basal ganglia. II. The place of subthalamic nucleus and external pallidium in basal ganglia circuitry. Brain Res Rev 1995; 20:128-54. https://doi.org/10.1016/0165-0173(94)00008-D.
21. Bolam J P, Hanley J J, Booth Pa C, Bevan M D. Synaptic organisation of the basal ganglia. J Anat 2000; 196:527-42. https://doi.org/10.1046/j.1469-7580.2000.19640527.x.
22. Baufreton J, Kirkham E, Atherton J F, Menard A, Magill P J, Bolam J P, et al. Sparse but selective and potent synaptic transmission from the globus pallidus to the subthalamic nucleus. J Neurophysiol 2009; 102:532-45. https://doi.org/10.1152/jn.00305.2009.
23. Nambu A, Tokuno H, Takada M. Functional significance of the corticoesubthalamoepallidal ‘hyperdirect’ pathway. Neurosci Res 2002; 43:111-7. https://doi.org/10.1016/S0168-0102(02)00027-5.
24. Rinvik E, Ottersen O P. Terminals of subthalamonigral fibres are enriched with glutamate-like immunoreactivity: an electron microscopic, immunogold analysis in the cat. J Chem Neuroanat 1993; 6:19-30. https://doi.org/10.1016/0891-0618(93)90004-N.
25. Kita T, Kita H. The subthalamic nucleus is one of multiple innervation sites for long-range corticofugal axons: a single-axon tracing study in the rat. J Neurosci 2012; 32:5990-9. https://doi.org/10.1523/JNEUROSCI.5717-11.2012.
26. Markram H, Muller E, Ramaswamy S, Reimann M W, Abdellah M, Sanchez C A, et al. Reconstruction and simulation of neocortical microcircuitry. Cell 2015; 163:456-92. https://doi.org/10.1016/j.cell.2015.09.029.
27. Destexhe A, Rudolph M, Fellous J-M, Sejnowski T J. Fluctuating synaptic conductances recreate in vivo-like activity in neocortical neurons. Neuroscience 2001; 107:13-24. https://doi.org/10.1016/S0306-4522(01)00344-X.
28. Milosevic L, Kalia S K, Hodaie M, Lozano A M, Popovic M R, Hutchison W D. Physiological mechanisms of thalamic ventral intermediate nucleus stimulation for tremor suppression. Brain 2018; 141:2142-55. https://doi.org/10.1093/brain/awy139.
29. Levy R, Lozano A M, Hutchison W D, Dostrovsky J O. Dual microelectrode technique for deep brain stereotactic surgery in humans. Oper. Neurosurg. (Hagerstown) 2007; 60. https://doi.org/10.1227/01.NEU.0000255389.85161.03. ONS-277-ONS-284.
30. Hutchison W D, Allan R J, Opitz H, Levy R, Dostrovsky J O, Lang A E, et al. Neurophysiological identification of the subthalamic nucleus in surgery for Parkinson's disease. Ann Neurol 1998; 44:622-8. https://doi.org/10.1002/ana.410440407.
31. Basha D, Dostrovsky J O, Lopez Rios A L, Hodaie M, Lozano A M, Hutchison W D. Beta oscillatory neurons in the motor thalamus of movement disorder and pain patients. Exp Neurol 2014; 261:782-90. https://doi.org/10.1016/j.expneurol.2014.08.024.
32. Milosevic L, Kalia S K, Hodaie M, Lozano A M, Fasano A, Popovic M R, et al. Neuronal inhibition and synaptic plasticity of basal ganglia neurons in Parkinson's disease. Brain 2018; 141:177-90. https://doi.org/10.1093/brain/awx296.
33. Milosevic L, Kalia S K, Hodaie M, Lozano A, Popovic M R, Hutchison W. Subthalamic suppression defines therapeutic threshold of deep brain stimulation in Parkinson's disease. J Neurol Neurosurg Psychiatry 2019; 90:1105-8.https://doi.org/10.1136/jnnp-2019-321140.
34. Bar-Gad I, Elias S, Vaadia E, Bergman H. Complex locking rather than complete cessation of neuronal activity in the globus pallidus of a 1-methyl-4-phenyl-1,2,3,6-tetrahydropyridine-treated primate in response to pallidal microstimulation. J Neurosci 2004; 24:7410-9. https://doi.org/10.1523/JNEUROSCI.1691-04.2004.
35. McIntyre C C, Savasta M, Kerkerian-Le Goff L, Vitek J L. Uncovering the mechanism(s) of action of deep brain stimulation: activation, inhibition, or both. Clin Neurophysiol 2004; 115:1239-48. https://doi.org/10.1016/j.clinph.2003.12.024.
36. Steiner L A, Tom as F J B, Planert H, Alle H, Vida I, Geiger J R P. Connectivity and dynamics underlying synaptic control of the subthalamic nucleus. J Neurosci 2019; 39:2470-81. https://doi.org/10.1523/JNEUROSCI.1642-18.2019.
37. Anderson T, Hu B, Pittman Q, Kiss Z H T. Mechanisms of deep brain stimulation: an intracellular study in rat thalamus. J Physiol 2004; 559:301-13. https://doi.org/10.1113/jphysiol.2004.064998.
38. McIntyre C C, Grill W M, Sherman D L, Thakor N V. Cellular effects of deep brain stimulation: model-based analysis of activation and inhibition. J Neurophysiol 2004; 91:1457-69. https://doi.org/10.1152/jn.00989.2003.
39. Albin R L, Young A B, Penney J B. The functional anatomy of basal ganglia disorders. Trends in Neurosciences 1989; 12:366-75. https://doi.org/10.1016/0166-2236(89)90074-X.
40. Alexander G E, Crutcher M D. Functional architecture of basal ganglia circuits: neural substrates of parallel processing. Trends in Neurosciences 1990; 13:266-71. https://doi.org/10.1016/0166-2236(90)90107-L.
41. DeLong M R. Primate models of movement disorders of basal ganglia origin. Trends in Neurosciences 1990; 13:281-5. https://doi.org/10.1016/0166-2236(90)90110-V.
42. Surmeier D J, Ding J, Day M, Wang Z, Shen W. D1 and D2 dopamine-receptor modulation of striatal glutamatergic signaling in striatal medium spiny neurons. Trends in Neurosciences 2007; 30:228-35. https://doi.org/10.1016/j.tins.2007.03.008.
43. Louis E D, Faust P L, Vonsattel J-P G, Honig L S, Rajput A, Robinson C A, et al. Neuropathological changes in essential tremor: 33 cases compared with 21 controls. Brain 2007; 130:3297-307. https://doi.org/10.1093/brain/awm266.
44. Louis E D, Kerridge C A, Chatterjee D, Martuscello R T, Diaz D T, Koeppen A H, et al. Contextualizing the pathology in the essential tremor cerebellar cortex: a pathologomics approach. Acta Neuropathol 2019; 138:859-76. https://doi.org/10.1007/s00401-019-02043-7.
45. Axelrad J E, Louis E D, Honig L S, Flores I, Ross G W, Pahwa R, et al. Reduced Purkinje Cell Number in Essential Tremor: A Postmortem Study. Arch Neurol 2008; 65:101-7. https://doi.org/10.1001/archneurol.2007.8.
46. Shill H A, Adler C H, Sabbagh M N, Connor D J, Caviness J N, Hentz J G, et al. Pathologic findings in prospectively ascertained essential tremor subjects. Neurology 2008; 70:1452. https://doi.org/10.1212/01.wnl.0000310425.76205.02.
47. Louis E D, Faust P L, Ma K J, Yu M, Cortes E, Vonsattel J-P G. Torpedoes in the Cerebellar Vermis in Essential Tremor Cases vs. Controls. Cerebellum 2011; 10:812-9. https://doi.org/10.1007/s12311-011-0291-0.
48.Yu M, Ma K, Faust P L, Honig L S, Cortes E, Vonsattel J-P G, et al. Increased number of Purkinje cell dendritic swellings in essential tremor. European Journal of Neurology 2012; 19:625-30. https://doi.org/10.1111/j.1468-1331.2011.03598.x.
49. Paris-Robidas S, Brochu E, Sintes M, Emond V, Bousquet M, Vandal M, et al. Defective dentate nucleus GABA receptors in essential tremor. Brain 2012; 135:105-16. https://doi.org/10.1093/brain/awr301.
50. Helmich R C, Toni I, Deuschl G, Bloem B R. The Pathophysiology of Essential Tremor and Parkinson's Tremor. Current Neurology and Neuroscience Reports 2013; 13. https://doi.org/10.1007/s11910-013-0378-8.
51. Kane A, Hutchison W D, Hodaie M, Lozano A M, Dostrovsky J O. Enhanced synchronization of thalamic theta band local field potentials in patients with essential tremor. Experimental Neurology 2009; 217:171-6. https://doi.org/10.1016/j.expneurol.2009.02.005.
52. Lenz F A, Tasker R R, Kwan H C, Schnider S, Kwong R, Murayama Y, et al. Single unit analysis of the human ventral thalamic nuclear group: correlation of thalamic “tremor cells” with the 3-6 Hz component of parkinsonian tremor. J Neurosci 1988; 8:754-64.
53. Barnikol U B, Popovych O V, Hauptmann C, Sturm V, Freund H-J, Tass P A. Tremor entrainment by patterned low-frequency stimulation. Philosophical Transactions of the Royal Society A: Mathematical, Physical and Engineering Sciences 2008; 366:3545-73. https://doi.org/10.1098/rsta.2008.0104.
54. Swan B D, Brocker D T, Hilliard J D, Tatter S B, Gross R E, Turner D A, et al. Short pauses in thalamic deep brain stimulation promote tremor and neuronal bursting. Clinical Neurophysiology 2016; 127:1551-9. https://doi.org/10.1016/j.clinph.2015.07.034.
55. Lymer J, Prescott I A, Levy R. Microstimulation-induced inhibition of thalamic reticular nucleus in non-human primates. Exp Brain Res 2019; 237:1511-20. https://doi.org/10.1007/s00221-019-05526-x.
56. Ulrich D, Huguenard J R. Purinergic inhibition of GABA and glutamate release in the thalamus: Implications for thalamic network activity. Neuron 1995; 15:909-18. https://doi.org/10.1016/0896-6273(95)90181-7.
57. Schlag J, Waszak M. Electrophysiological properties of units of the thalamic reticular complex. Experimental Neurology 1971; 32:79-97. https://doi.org/10.1016/0014-4886(71)90167-1.
58. Landisman C E, Long M A, Beierlein M, Deans M R, Paul D L, Connors B W. Electrical Synapses in the Thalamic Reticular Nucleus. J Neurosci 2002; 22:1002-9. https://doi.org/10.1523/JNEUROSCI.22-03-01002.2002.
59. Gentet L J, Ulrich D. Electrophysiological characterization of synaptic connections between layer V I cortical cells and neurons of the nucleus reticularis thalami in juvenile rats. European Journal of Neuroscience 2004; 19:625-33. https://doi.org/1 s0.1111/j.1460-9568.2004.03168.x.
60. Gentet L J, Ulrich D. Strong, reliable and precise synaptic connections between thalamic relay cells and neurons of the nucleus reticularis in juvenile rats. The Journal of Physiology 2003; 546:801-11. https://doi.org/10.1113/jphysiol.2002.032730.
61. Bazhenov M, Timofeev I, Steriade M, Sejnowski T J. Self-sustained rhythmic activity in the thalamic reticular nucleus mediated by depolarizing GABA A receptor potentials. Nature Neuroscience 1999; 2:168-74. https://doi.org/10.1038/5729.
62. Nanobashvili Z, Chachua T, Nanobashvili A, Bilanishvili I, Lindvall O, Kokaia Z. Suppression of limbic motor seizures by electrical stimulation in thalamic reticular nucleus. Experimental Neurology 2003; 181:224-30. https://doi.org/10.1016/S0014-4886(03)00045-1.
63. Nanobashvili Z I, Surmava A G, Bilanishvili I G, Barbaqadze M G, Mariamidze M D, Khizanishvili N A. Significance of the Thalamic Reticular Nucleus GABAergic Neurons in Normal and Pathological Activity of the Brain, 2012. https://doi.org/10.4236/jbbs.2012.24051.
64. Pantoja-Jiménez C R, Magdaleno-Madrigal V M, Almazán-Alvarado S, Fernández-Mas R. Anti-epileptogenic Effect of High-frequency Stimulation in the Thalamic Reticular Nucleus on PTZ-induced Seizures. Brain Stimulation 2014; 7:587-94. https://doi.org/10.1016/j.brs.2014.03.012.
65. Chang W J, Chang W P, Shyu B C. Suppression of cortical seizures by optic stimulation of the reticular thalamus in P V-mhChR2-YFP BAC transgenic mice. Molecular Brain 2017; 10:42. https://doi.org/10.1186/s13041-017-0320-0.
66. Wang Z, Wang Q. Eliminating Absence Seizures through the Deep Brain Stimulation to Thalamus Reticular Nucleus. Front Comput Neurosci 2017; 11. https://doi. org/10.3389/fncom 0.2017.00022.
67. Blumenfeld H. Cellular and Network Mechanisms of Spike-Wave Seizures. Epilepsia 2005; 46:21-33. https://doi.org/10.1111/j.1528-1167.2005.00311.x.
68. Krauss G L, Koubeissi M Z. Cerebellar and thalamic stimulation treatment for epilepsy. In: Sakas D E, Simpson B A, editors. Operative Neuromodulation: Volume 2: Neural Networks Surgery, Vienna: Springer; 2007, p. 347-56. https://doi.org/10.1007/978-3-211-33081-4_40.
69. Steriade M, Parent A, Hada J. Thalamic projections of nucleus reticularis thalami of cat: A study using retrograde transport of horseradish peroxidase and fluorescent tracers. Journal of Comparative Neurology 1984; 229:531-47. https://doi.org/10.1002/cne.902290407.
70. Kultas-Ilinsky K, Yi H, Ilinsky I A. Nucleus reticularis thalami input to the anterior thalamic nuclei in the monkey: a light and electron microscopic study. Neuroscience Letters 1995; 186:25-8. https://doi.org/10.1016/0304-3940(95)11273-Y.
71. Cox C L, Huguenard J R, Prince D A. Nucleus reticularis neurons mediate diverse inhibitory effects in thalamus. PNAS 1997; 94:8854-9. https://doi.org/10.1073/pnas.94.16.8854.
72. Kim U, Sanchez-Vives M V, McCormick D A. Functional Dynamics of GABAergic Inhibition in the Thalamus. Science 1997; 278:130-4. https://doi.org/10.1126/science.278.5335.130.
73. Yamamoto T, Katayama Y. Deep brain stimulation therapy for the vegetative state. Neuropsychological Rehabilitation 2005; 15:406-13. https://doi.org/10.1080/09602010443000353.
74. Chudy D, Deletis V, Almahariq F, Marčinković P, Škrlin J, Paradžik V. Deep brain stimulation for the early treatment of the minimally conscious state and vegetative state: experience in 14 patients. Journal of Neurosurgery 2018; 128:1189-98. https://doi.org/10.3171/2016.10.JNS161071.
75. Lim A S, Moro E, Lozano A M, Hamani C, Dostrovsky J O, Hutchison W D, et al. Selective enhancement of rapid eye movement sleep by deep brain stimulation of the human pons. Annals of Neurology 2009; 66:110-4. https://doi.org/10.1002/ana.21631.
76. Arnulf I, Ferraye M, Fraix V, Benabid A L, Chabardès S, Goetz L, et al. Sleep induced by stimulation in the human pedunculopontine nucleus area. Annals of Neurology 2010; 67:546-9. https://doi.org/10.1002/ana.21912.
77. McAlonan K, Cavanaugh J, Wurtz R H. Guarding the gateway to cortex with attention in visual thalamus. Nature 2008; 456:391-4. https://doi.org/10.1038/nature07382.
78. Lewis L D, Voigts J, Flores F J, Schmitt L I, Wilson M A, Halassa M M, et al. Thalamic reticular nucleus induces fast and local modulation of arousal state. ELife 2015; 4:e08760. https://doi.org/10.7554/eLife.08760.

1. A device for performing one or more functions related to deep brain stimulation (DBS) of a target structure, wherein the device comprises:
a memory unit storing program instructions for performing one or more functions related to DBS and a target model corresponding to the target structure, the target model including a neuronal model for modeling one or more neurons of the target structure and a short-term plasticity model for modeling temporal behaviour of the neuron in response to one or more stimulus pulses over time;
one or more processors that are coupled to the memory unit, the one or more processors, when executing the program instructions, being configured to:
receive input data related to the target structure including a brain region of the target structure, stimulus parameters including pulse frequency, and model parameters that correspond to the brain structure;
determine a neuronal response of the target structure by applying the stimulus parameters and the model parameters to the target model; and
perform any combination of outputting the neuronal response, storing the neuronal response and transmitting the neuronal response to another device.

2. The device of claim 1, wherein the model parameters include a proportion of inhibitory inputs and exhibitory inputs for the target structure.

3. The device of claim 2, wherein the neuron model comprises a leaky integrate and fire (LIF) single neuron model.

4. The device of claim 3, wherein the short-term plasticity model comprises the Tsodyks-Markram (TM) model.

5. The device of claim 3, wherein the target model further a background synaptic activity model that is added to the neuron model to reproduce an impact of synaptic noise, the background synaptic activity model being implemented using an Ornstein-Uhlenbeck process.

6. The device of claim 2, wherein the memory unit stores determination program instructions that, when executed by the one or more processors, configure the one or more processors for determining one or more of the stimulus parameters, including frequency, generating inhibitory and excitatory synaptic responses and scaling the inhibitory and excitatory synaptic responses with respect to weights representing the proportion of inhibitory and exhibitory inputs.

7. The device of claim 2, wherein the memory unit stores stimulation program instructions that, when executed by the one or more processors, configure the one or more processors for controlling one or more stimulation device(s) to deliver DBS to a patient where stimulus frequency is selected based on the target structure and desired treatment for the patient.

8. The device of claim 7, wherein when there are more inhibitory inputs than exhibitory inputs associated with the target structure, the one or more processors, when executing the stimulation instructions, are configured to:
control the one or more stimulation devices to deliver a low frequency stimulation when partial suppression of neuronal output is desired; or
control the one or more stimulation devices to deliver a high frequency stimulation when complete suppression of neuronal output is desired.

9. The device of claim 7, wherein when there are more exhibitory inputs than inhibitory inputs associated with the target structure, the one or more processors, when executing the stimulation instructions, are configured to:
control the one or more stimulation devices to deliver a low frequency stimulation when upregulation of neuronal activity is determined; or
control the one or more stimulation devices to deliver a high frequency stimulation when downregulation of neuronal activity is desired.

10. The device of claim 7, wherein the DBS stimulus is provided to the patient using an open loop stimulation mode or a closed loop stimulation mode.

11. A method for performing one or more functions related to deep brain stimulation (DBS) of a target structure, wherein the method is performed by one or more processors and the method comprises:
obtaining a target model corresponding to the target structure, the target model including a neuronal model for modeling one or more neurons of the target structure and a short-term plasticity model for modeling temporal behaviour of the neuron in response to one or more stimulus pulses over time;
receiving input data related to the target structure including a brain region of the target structure, stimulus parameters including pulse frequency, and model parameters that correspond to the brain structure;
determining a neuronal response of the target structure by applying the stimulus parameters and the model parameters to the target model; and
performing any combination of outputting the neuronal response, storing the neuronal response and transmitting the neuronal response to another device.

12. The method of claim 11, wherein the model parameters include a proportion of inhibitory inputs and exhibitory inputs for the target structure.

13. The method of claim 12, wherein the neuron model comprises a leaky integrate and fire (LIF) single neuron model.

14. The method of claim 13, wherein the short-term plasticity model comprises the Tsodyks-Markram (TM) model.

15. The method of claim 13, wherein the target model further a background synaptic activity model that is added to the neuron model to reproduce an impact of synaptic noise, the background synaptic activity model being implemented using an Ornstein-Uhlenbeck process.

16. The method of claim 12, wherein the method comprises determining one or more of the stimulus parameters, including frequency, generating inhibitory and excitatory synaptic responses and scaling the inhibitory and excitatory synaptic responses with respect to weights representing the proportion of inhibitory and exhibitory inputs.

17. The method of claim 12, wherein the method comprises controlling one or more stimulation device(s) to deliver DBS to a patient where stimulus frequency is selected based on the target structure and desired treatment for the patient and when there are more inhibitory inputs than exhibitory inputs associated with the target structure, the method further comprises:
controlling the one or more stimulation devices to deliver a low frequency stimulation when partial suppression of neuronal output is desired; or
controlling the one or more stimulation devices to deliver a high frequency stimulation when complete suppression of neuronal output is desired.

18. The method of claim 12, wherein the method comprises controlling one or more stimulation device(s) to deliver DBS to a patient where stimulus frequency is selected based on the target structure and desired treatment for the patient and when there are more exhibitory inputs than inhibitory inputs associated the method further comprises:
controlling the one or more stimulation devices to deliver a low frequency stimulation when upregulation of neuronal activity is desired; or
controlling the one or more stimulation devices to deliver a high frequency stimulation when downregulation of neuronal activity is desired.

19. A computer readable medium comprising a plurality of instructions that are executable on one or more processors of a device for configuring the one or more processors to implement a method for at least one function related to deep brain stimulation, wherein the method is defined according to claim 11.

20. A method for delivering deep brain stimulation, comprising:
receiving input data for a target structure;
determining whether there are more inhibitory or exhibitory inputs associated with the received target structure;
where there are more inhibitory inputs:
determining whether to partially or completely suppress neuronal output;
where partial suppression of neuronal output is determined,
delivering a low frequency stimulation; and
where complete suppression of neuronal output is determined,
delivering a high frequency stimulation; and
where there are more exhibitory inputs:
determining whether to upregulate or downregulate neuronal activity;
where upregulation of neuronal activity is determined, delivering a low frequency stimulation; and
where downregulation of neuronal activity is determined, delivering a high frequency stimulation.
